(19) United States
(12) Patent Application Publication
(10) Pub. No.: US 2020/0268684 A1
(43) Pub. Date: Aug. 27, 2020

(54) COMPOSITION FOR TREATING AND PREVENTING NEUROLOGICAL DISEASES, NEUROINFLAMATION AND ALZHEIMER'S DISEASE

(71) Applicants: Joshua M. costin, Naples, FL (US); John M. WILLIAMS, Bonita Springs, FL (US); Norman RELKIN, Harrington Park, NJ (US); HSRX GROUP, LLC, Tucson, AZ (US)

(72) Inventors: Joshua M COSTIN, Naples, FL (US); John M WILLIAMS, Bonita Springs, FL (US); Norman RELKIN, Harrington Park, NJ (US); Dan LI, Fort Meyer, FL (US)

(21) Appl. No.: 16/063,137

(22) PCT Filed: Dec. 16, 2016
(86) PCT No.: PCT/IB16/57741

§ 371 Date: Jun. 15, 2018

Related U.S. Application Data
(60) Provisional application No. 62/268,371, filed on Dec. 16, 2015.
(60) Provisional application No. 62/345,375, filed on Jun. 3, 2016.

(51) Int. Cl. A61K 31/12; A61K 36/9066; A61P 25/28; A61P 29/00; A61K 49/00; A61K 31/445; A61K 31/473; A61K 31/55; A61K 31/27; A61K 31/13

(52) U.S. Cl. CPC A61K 31/12; A61K 36/9066; A61P 25/28; A61P 29/00; A61K 31/13; A61K 31/445; A61K 31/473; A61K 31/55; A61K 31/27; A61K 49/0002

(57) ABSTRACT

The present invention relates generally to compositions and methods of use that include compounds that treat and prevent neurological disorders including Alzheimer's disease, neuroinflammation, and diseases and conditions associated with protein misfolding and/or protein aggregation.

### CROSS-REFERENCE TO RELATED APPLICATIONS

This application claims the benefit of U.S. Provisional Application No. 62/268,371, filed Dec. 16, 2015, and U.S. Provisional Application No. 62/345,375, filed Jun. 3, 2016. The contents of the referenced applications are incorporated into the present application by reference.

### BACKGROUND OF THE INVENTION

### A. Field of the Invention

The present invention relates to formulations containing a mixture of compounds capable of preventing and treating neurological diseases, protein misfolding, protein aggregation, neuroinflammation, and Alzheimer's disease.

### B. Description of Related Art

Many human neurodegenerative disorders have been linked to the aggregation of proteins that are subject to pathologic misfolding (Ellisdon et al., 2004). Proteins have a native structure that imparts stability, functionality, and specificity to their interactions with other molecules. Genetic alterations in proteins, post-translational modifications, and exposure to certain environmental conditions can alter the three dimensional structure of proteins leading to their assumption of misfolded configurations. In some cases, misfolding creates an energetically unfavorable configuration that promotes self-assembly of the protein into aggregates. Aggregated misfolded proteins can be especially toxic to many cells, including neurons. The penetration of aggregated misfolded proteins into cell membranes can result in cell death. Protein misfolding has been implicated in human neurodegenerative disorders such as Alzheimer's disease (beta-amyloid and phosphorylated tau proteins), Parkinson's disease (alpha-synuclein protein), Dementia with Lewy bodies (beta-amyloid, phosphorylated tau and alpha-synuclein proteins), Frontotemporal dementias (tau protein), Spnngifnrm encephalopathies (prion protein), as well as in many other central and systemic amyloidoses (see Ellisdon et al., 2004). Interventions that reduce propensity for protein misfolding, disaggregate or otherwise alter aggregation pathways, and/or mitigate the toxicity of misfolded proteins and their aggregates represent potential means of preventing and treating Alzheimer's disease and several other human disorders involving protein misfolding.

Alzheimer's disease (AD) is one disease in which protein misfolding is implicated. AD is the leading cause of dementia in the elderly (Mayo Clinic, Alzheimer's, 2014). AD is estimated to afflict over 5 million Americans and is rapidly increasing in prevalence as the population ages. Although its symptoms can be treated, AD remains an incurable and fatal disorder. The causes of AD are not completely known, but genetic and environmental factors have been implicated in its pathogenesis. The underlying pathologic processes evolve over several decades of life. Among the earliest documented pathologic changes is accumulation of soluble and insoluble amyloid aggregates in the brain and brain vasculature related to abnormal beta amyloid production, aggregation, metabolism and/or clearance. Chronic neuro-inflammation occurs in AD and is associated with aberrant microglial activation as well as cytokine and chemokine alterations. Increased oxidative damage to neurons by reactive oxygen species and advanced glycation end products occurs in AD. AD is also associated with hyper-phosphorylation of tau and tau aggregation, leading to neurofibrillary tangle formation. As AD pathology progresses, there is significant synaptic and neuronal loss as well as gliosis resulting in brain atrophy. Deficits in neurotransmitters such as acetylcholine and glutamine as well as disturbances of brain glucose metabolism also occur. AD manifests as dementia with a progressive decline in cognition, daily function and behavior, typically affecting short term recall initially and progressing to affect all cognitive domains. (Mayo Clinic, Alzheimer's, 2014).

Inflammation may be another underlying factor in AD as well as several other neurodegenerative diseases. There are multiple proteins involved in inflammatory pathways, such as the cyclo-oxygenase enzymes COX1 and COX2 and the 5-lipoxygenase enzyme 5LOX. Inhibition of these enzymes or downstream inflammatory intermediates such as leukotrienes can suppress inflammation. Pro-inflammatory cytokines such as IFNγ. TNF-alpha, IL-1 and IL-6 are produced in T-helper type 1 (Th1) responses that may foster inflammation in the AD brain. Reduction of pro-inflammatory cytokines or increases in anti-inflammatory cytokines could have therapeutic benefits in AD. Some inhibitors of inflammation include NSAIDs and anti-inflammatory cytokines such IL-2 and T-helper 2 (Th2) response cytokines such as IL-4. Past clinical trials testing COX-1 inhibitors and other NSAIDs as potential treatments or preventions for AD have largely failed. It has been hypothesized that other COX inhibitors, especially in conjunction with LOX inhibitors (e.g. dual COX/LOX inhibitors), may provide an improved therapeutic approach to the treatment of aging-related brain disorders such as Alzheimer's disease and have acceptable gastrointestinal tolerability (Hoozemans et al., 2008). In this regard, COX2 inhibitors have been shown to be a potential therapy for neuronal inflammation by significantly reversing aging-induced retention deficits in mice (Bishnoi et al., 2005).

Reduction of oxidative damage may also decrease neuroinflammation involved in AD and other neurodegenerative diseases. As an example, reduction of COX1, COX2, and 5LOX activity reduces inflammation in part through mitigation of oxidized fatty acids. Specifically, COX1 and COX2 reduce neurotoxicity and neurodegeneration through mitigation of oxidation products of two fatty acids, arachidonic acid (AA) and docosahexaenoic acid (DHA) (Hoozemans et al.). Pharmacoepidemiological data, analytical data from human tissue and body fluids, and mechanistic data mostly from murine models all have implicated AA and DHA oxidation products in the pathogenesis of neurodegeneration (Hoozemans et al., 2008). The 5-LOX enzyme is mainly involved in the conversion of arachidonic acid into inflammatory mediators. While cyclooxygenase (COX1 and COX2) enzymes generate prostaglandins, 5-LOX generates leukotrienes (Silverman et al., 1999). Inhibition of COX1 and COX2 shunts arachidonic acid to the 5-LOX pathway thus producing leukotrienes, molecules involved in the inflammatory and allergic response (Steinhilber et al., 2013).

Current treatments for AD include those that assist in boosting levels of cell-to-cell communication, such as acetylcholinesterase inhibitors and memantine. (Mayo Clinic, Alzheimer's, 2014). However, despite extensive research, it has been over a decade since a new class of medications was approved for AD. No dietary supplement has ever been found to be beneficial for human AD patients in large-scale, well-controlled prospective clinical trials.

Curcumin (diferulomethane) is a polyphenolic phytochemical found in turmeric root that has anti-oxidant, anti-inflammatory, anti-amyloid, and other properties. Curcumin is a major ingredient in curry powder. There is evidence that consumption of curry may lower the incidence of dementia (Ng, 2006). Curcumin's many possible benefits have been demonstrated in preclinical studies over the past two decades showing encouraging effects of curcumin on amyloid precursor protein metabolism, beta-amyloid aggregates, tau-containing neurofibrillary tangles, neuro-inflammation and several other elements of AD pathology (Lim, 2001; Yang, 2005; Ma, 2013).

Further, in vitro, in vivo, and/or clinical studies show that curcumin has the potential to treat many other diseases, conditions, disorders, causes of such, and/or symptoms of such. For example, curcumin has antimicrobial and antiviral effects, is a powerful anti-inflammatory and immunomodulatory agent, protects the cardiovascular system, is a cancer chemopreventative and chemotherapeutic agent, is neuroprotective and neurotherapeutic, is a potential drug for diabetic and obesity pharmacology, protects against renal injury, protects the lungs following cardiopulmonary bypass, is an agent for treatment of gastrointestinal disorders, is a modulator of wound healing, is a reproductive system modulator, is an anti-angiogenic, and is an anti-toxicological agent, among other things (Beevers 2011).

However, the pharmacokinetics associated with curcumin pose a significant challenge to widespread clinical use of curcumin for the treatment of many human disease as curcumin exhibits extremely poor gastrointestinal absorption and oral bioavailability (Beevers 2011). Further, a major obstacle to the oral administration of purified curcumin to humans is that nearly 100% of ingested material is converted into an inactive glucuronidated form that does not cross the blood brain barrier. This conversion may be the reason that promising in vitro and animal studies with curcumin have not correlated with efficacy in human subjects (Ringman, 2005). In one study (Ringman, 2012), investigators gave AD patients up to 4 grams per day of a purified curcumin preparation, roughly equivalent to 32 times the average individual's dietary intake in India. Despite the administration of such high doses, very little (approximately 7.76 ng/ml) of curcumin was found in the patients' blood and no unmodified curcumin was detected in cerebrospinal fluid (CSF). Id. Further, no alterations in AD-associated CSF biomarkers were observed following curcumin ingestion. Id.

Recent efforts to develop more bio-available curcumin preparations have used liposome encapsulation techniques or added piperines to curcumin preparations. (Ringman, 2005). However, few if any attempts have been successful in providing significant levels of curcumin in the blood (Ringman 2012; Ringman, 2005). For example, blood and urine levels of curcumin are either non-existent or barely measurable with oral doses of pure curcumin below 2000 mg (Ringman, 2005). Further, to date, no orally administered formulation has been shown to produce detectable levels of curcumin in the cerebrospinal fluid.

An additional obstacle to the oral administration of purified curcumin to humans is the gastrointestinal side effects. The side effects can include diarrhea, black stools, gastric irritation, and nausea (Ringman 2005; Ringman 2012). These side effects have been sufficiently severe in some patients to cause those patients to withdraw from clinical trials (Ringman 2012).

One proprietary composition which contains curcumin, HSRx-888, was tested in an in vitro and animal (mice) model of Alzheimer's disease. It was found to reduce amyloid plaque burden and have other positive effects on AD-related brain pathology (Shytle et al., 2009, Shytle et al., 2012). However, activity in vitro and in rodents do not always correlate with activity in humans or uptake of curcumin in humans, likely due in part to the different pharmacokinetic profiles of these models and humans.

### SUMMARY OF THE INVENTION

The present invention provides a solution to the current problems facing treatment and prevention of Alzheimer's disease, inflammation, neuroinflammation, diseases and conditions that are caused by neuroinflammation, protein misfolding, protein aggregation, and diseases and conditions that are caused by protein misfolding and protein aggregation.

The inventors have surprisingly determined that a combination of several compounds found in turmeric can prevent and treat Alzheimer's disease, inflammation, protein misfolding, protein aggregation, and can increase uptake of curcumin in human subjects. The inventors have also determined that specific relative concentrations of the compounds enhance these abilities of the combined compounds. In addition, the inventors have determined that using compounds of the present invention with additional agents for treating or preventing disease and conditions such as Alzheimer's disease, inflammation, and protein misfolding/aggregation related diseases and conditions enhance the ability of the combined compounds to prevent and treat such diseases and conditions. Further, the combinations disclosed herein provide benefits in treatment and/or prevention of other neurological disorders, diseases, and conditions such as other degenerative/protein misfolding disorders, cerebrovascular diseases, inflammatory diseases, trauma/closed head injuries, epilepsies, and/or neoplasms.

In one aspect, disclosed is a composition of any one of, any combination of, or all of biomarkers 1-16, 18, 19 disclosed herein and curcumin. In some instances the composition includes curcumin and/or a functional derivative of curcumin and biomarker 1 having an accurate mass of 120.094 amu and having a relative abundance of at least 2.17%, wherein the biomarker 1 is found in Curcuma longa, and wherein the relative abundance is relative to 25 mg/ml salicylic acid spiked in 0.5 mg/ml of the composition dissolved in ethanol.

In some embodiments, the composition further includes any one of, or any combination of, or all of the following additional biomarkers: biomarker 2 having an accurate mass of 134.110 amu and having a relative abundance of at least 0.31%; biomarker 6 having an accurate mass of 200.157 amu and having a relative abundance of at least 0.47%; and biomarker 12 having an accurate mass of 232.146 amu and having a relative abundance of at least 2.38%, wherein the biomarkers are found in Curcuma longa, and wherein the relative abundance is relative to 25 mg/ml salicylic acid spiked in 0.5 mg/ml of the composition dissolved in ethanol. In some embodiments, the composition has at least 2, 3, or 4 of biomarkers 1, 2, 6, and 12.

In some embodiments, the composition disclosed herein further includes one or more of: biomarker 3 having an accurate mass of 150.104 amu and having a concentration of at least 0.04% by weight; biomarker 4 having an accurate mass of 176.120 amu and having a relative abundance of at least 0.96%; biomarker 5 having an accurate mass of 192.091 amu and having a relative abundance of at least 1.74%; biomarker 7 having an accurate mass of 202.172 amu and having a relative abundance of at least 0.87%; biomarker 8 having an accurate mass of 204.188 amu and having a relative abundance of at least 0.30%; biomarker 9 having an accurate mass of 216.151 amu and having a relative abundance of at least 10.75%; biomarker 10 having an accurate mass of 218.203 amu and having a relative abundance of at least 4.00%; biomarker 11 having an accurate mass of 220.183 amu and having a relative abundance of at least 0.72%; biomarker 13 having an accurate mass of 234.162 amu and having a relative abundance of at least 3.52%; biomarker 14 having an accurate mass of 256.240 amu and having a relative abundance of at least 0.25%; biomarker 15 having an accurate mass of 308.105 amu and having a concentration of at least 1.50% by weight; biomarker 16 having an accurate mass of 338.115 amu and having a concentration of at least 1.67% by weight; biomarker 18 having an accurate mass of 372.157 amu and having a concentration of at least 0.88% by weight; and biomarker 19 having an accurate mass of 450.261 amu and having a relative abundance of at least 0.61%, wherein each biomarker is found in Curcuma longa, and wherein the relative abundance is relative to 25 mg/ml salicylic acid spiked in 0.5 mg/ml of the composition dissolved in ethanol.

In some embodiments, the mass of each biomarker is the mass as determined by a Direct Analysis in Real Time-TOF (DART-TOF) mass spectrometer.

In some embodiments, at least one of the biomarker(s) are synthetically obtained. In some embodiments, at least one of the biomarker(s) are isolated from a plant. In one instance at least one of the biomarkers(s) are isolated from Curcuma longa. In some embodiments, the composition has an at least 90%, preferably at least 95%, or at least 98% batch-to-batch chemical consistency of relative abundance for the biomarkers.

In some embodiments, the composition further includes at least one drug. In some embodiments, the composition further includes at least one acetylcholinesterase inhibitor. In one instance at least one acetylcholinesterase inhibitor is donepezil, tacrine, galantamine, rivastigmine, salts thereof, or any combination thereof. In some embodiments, the composition further includes an N-methyl-D-aspartate (NMDA) receptor antagonist. In some embodiments, the NMDA receptor antagonist is memantine. In some embodiments, the composition further includes at least one anti-inflammatory drug. In one instance at least one anti-inflammatory drug is a nonsteroidal anti-inflammatory drug. In one instance the nonsteroidal anti-inflammatory drug is acetylsalicylic acid, ibuprofen, ketoprofen, or naproxen, salts thereof, or any combination thereof.

In some embodiments, the composition is formulated for intranasal administration. In one instance the composition is administered as a dry powder and/or by a nebulizer. In some embodiments, the composition is formulated for topical application, administration through injection, and/or oral administration. In one instance the composition is formulated for oral administration. In another instance the composition is a lozenge, a powder, a tablet, a gel-cap, a gelatin, a liquid solution, a food, in a food, and/or a dissolvable film.

In some embodiments, at least one of the biomarker(s) is capable of binding to amyloid. In some embodiments, at least one of the biomarker(s) is capable of preventing amyloid from aggregating. In some embodiments, the composition is formulated to decrease amyloid secretion. In some embodiments, the composition is formulated to decrease both soluble and insoluble amyloid levels.

In some embodiments, the composition is formulated to decrease tau. In some embodiments, the composition is formulated to decrease phosphorylated tau and/or phosphorylation of tau.

In some embodiments, the composition is formulated to decrease protein misfolding. In some embodiments, the composition is formulated to decrease protein aggregation.

In some embodiments, the composition is formulated to decrease neuro-inflammation. In some embodiments, the composition is formulated to increase the ratio of IL-4 to IL-2.

In some embodiments, the composition is formulated to increase cognition.

In some embodiments, the composition is formulated to inhibit COX1 and/or COX2 or a pathway thereof. In some embodiments, the composition is formulated to inhibit 5LOX or a pathway thereof. In some embodiments, the composition is formulated to have an antioxidant activity. In some embodiments, the composition is formulated to scavenge a free radical. In some embodiments, the composition is formulated to increase a Th2 response.

In some embodiments, the composition is formulated to inhibit or treat a neurological disease, disorder, and/or condition. In some embodiments, the composition is formulated to inhibit or treat a degenerative/protein misfolding disorder, cerebrovascular disease, inflammatory disease, trauma/closed head injury, epilepsy, and/or neoplasm. In some embodiments, the composition is formulated to inhibit or treat Alzheimer's disease, Parkinson's disease, a Lewy body disease, frontotemporal degeneration, progressive supranuclear palsy, amyotrophic lateral sclerosis, multisystem atrophy, cerebral amyloidosis, spinocerebellar atrophy, ischemic stroke, reperfusion injury, cerebral vasospasm, multiple sclerosis, CNS lupus, a concussion, a contusion, chronic traumatic encephalopathy, a generalized seizure disorder, a partial seizure disorder, a metastatic tumor, and/or a primary CNS tumor. In some embodiments, the composition is formulated to inhibit or treat Alzheimer's disease.

In some embodiments, the composition is formulated to prevent a neurological disease, disorder, and/or condition. In some embodiments, the composition is formulated to prevent a degenerative/protein misfolding disorder, cerebrovascular disease, inflammatory disease, trauma/closed head injury, epilepsy, and/or neoplasm. In some embodiments, the composition is formulated to prevent Alzheimer's disease, Parkinson's disease, a Lewy body disease, frontotemporal degeneration, progressive supranuclear palsy, amyotrophic lateral sclerosis, multisystem atrophy, cerebral amyloidosis, spinocerebellar atrophy, ischemic stroke, reperfusion injury, cerebral vasospasm, multiple sclerosis, CNS lupus, a concussion, a contusion, chronic traumatic encephalopathy, a generalized seizure disorder, a partial seizure disorder, a metastatic tumor, and/or a primary CNS tumor. In some embodiments, the composition is formulated to prevent Alzheimer's disease.

In some embodiments, the composition is formulated as an anti-nausea. In some embodiments, the composition is formulated to treat a side effect and/or adverse event associated with a subject taking at least one acetylcholinesterase inhibitor, NMDA receptor antagonist, and/or curcumin. In some embodiments, the composition is formulated to prevent a side effect and/or adverse event associated with a subject taking at least one acetylcholinesterase inhibitor, NMDA receptor antagonist, and/or curcumin.

In some embodiments, the composition is formulated to increase the uptake of curcumin and/or an analog thereof into a subject when compared to the uptake of curcumin and/or an analog thereof without any of biomarkers 1 through 16, 18, and/or 19.

In some embodiments, the composition further includes at least one turmerone and has a weight ratio of curcumin and/or an analog thereof to turmerones of between 0.5 to 0.9.

In some embodiments, the composition is formulated to provide at least 30% of the curcumin and/or functional derivative thereof present in the composition into the serum of a human administered the composition. In some embodiments, the composition is formulated to provide at least 10 mg of curcumin and/or functional derivative thereof into the serum of a human administered the composition. In some embodiments, the composition is formulated to provide a Tₘₐₓ for curcumin and/or functional derivative thereof of 20 to 120 minutes in the serum of a human subject after administration to the subject. In some embodiments, the composition is formulated to provide a Cₘₐₓ for curcumin and/or functional derivative thereof of at least 5 micromolar in the serum of a human subject after administration to the subject.

In some embodiments, the composition is formulated to provide a Tₘₐₓ for biomarker 1 of 5 to 120 minutes in the serum of a human subject after administration to the subject. In some embodiments, the composition is formulated to provide a Tₘₐₓ for biomarker 2 of 2 to 60 minutes in the serum of a human subject after administration to the subject. In some embodiments, the composition is formulated to provide a Tₘₐₓ for biomarker 6 of 10 to 180 minutes in the serum of a human subject after administration to the subject. In some embodiments, the composition is formulated to provide a Tₘₐₓ for biomarker 12 of 5 to 20 minutes in the serum of a human subject after administration to the subject.

In some embodiments, the composition is formulated to provide curcumin and/or functional derivative thereof present in the composition into the cerebrospinal fluid of a human administered the composition. In some embodiments, the composition is formulated to provide at least 1 mg of curcumin and/or functional derivative thereof into the cerebrospinal fluid of a human administered the composition. In some embodiments, the composition is formulated to provide at least one of the biomarker(s) 1 through 16, 18, or 19 into the cerebrospinal fluid of a human administered the composition.

In some embodiments, the composition further includes an imaging agent. In one instance the imaging agent is covalently bound to at least one of the biomarker(s) 1 through 16, 18, or 19. In another instance the imaging agent is not covalently bound to any of the biomarker(s) 1 through 16, 18, or 19.

Disclosed herein are methods of treating a subject. In some embodiments, the method is a method of treating a subject at risk for and/or having a neurological disease, condition, and/or disorder, by administering any one of the compositions disclosed herein to the subject, and wherein the neurological disease, condition, and/or disorder is ameliorated in the subject and/or the onset is delayed in comparison to the expected onset of the neurological disease, condition, and/or disorder if the patient had not been treated. In some embodiments, the neurological disease, condition, and/or disorder is a degenerative/protein misfolding disorder, a cerebrovascular disease, an inflammatory disease, a trauma/closed head injury, an epilepsy, and/or a neoplasm. In some embodiments, the neurological disease, condition, and/or disorder is Alzheimer's disease, Parkinson's disease, a Lewy body disease, frontotemporal degeneration, progressive supranuclear palsy, amyotrophic lateral sclerosis, multisystem atrophy, cerebral amyloidosis, spinocerebellar atrophy, ischemic stroke, reperfusion injury, cerebral vasospasm, multiple sclerosis, CNS lupus, a concussion, a contusion, chronic traumatic encephalopathy, a generalized seizure disorder, a partial seizure disorder, a metastatic tumor, and/or a primary CNS tumor. In some embodiments, the neurological disease, condition, and/or disorder is Alzheimer's disease.

In some embodiments, the method is a method for treating a subject at risk for Alzheimer's disease or having Alzheimer's disease. In some embodiments, the method includes administering any one of the compositions disclosed herein to the subject, wherein at least one symptom of Alzheimer's disease is ameliorated in the subject or the onset of Alzheimer's disease is delayed in comparison to the expected onset of Alzheimer's disease if the patient had not been treated. In some embodiments, the method includes wherein the subject is identified as having amyloid secretion, amyloid aggregation, tau hyperphosphorylation, neuro-inflammation, or decreased cognition, or any combination thereof.

In some embodiments, the methods disclosed herein include wherein the subject is administered a total amount of between 1 and 10,000 mg, between 10 and 5,000 mg, between 50 and 2,500 mg, or between 100 and 1,000 mg of the composition during a 24 hour period.

In some embodiments, the methods disclosed herein include wherein at least one of the biomarker(s) 1 through 16, 18, or 19 is synthetically obtained. In some embodiments, the method includes wherein at least one of the biomarker(s) 1 through 16, 18, or 19 is isolated from plant. In one instance, the method includes wherein at least one of the biomarker(s) is isolated from Curcuma longa. In some embodiments, the method includes wherein the composition has an at least 95% batch-to-batch chemical consistency of relative abundance for the biomarkers.

In some embodiments, the methods disclosed herein include wherein the composition further includes an acetylcholinesterase inhibitor. In one instance the method includes wherein the acetylcholinesterase inhibitor is donepezil, tacrine, galantamine, rivastigmine, salts thereof, or any combination thereof. In one instance the method includes wherein the acetylcholinesterase inhibitor is donepezil, a salt thereof, or any combination thereof. In some embodiments, the methods disclosed herein include wherein the composition further includes a N-methyl-D-aspartate (NMDA) receptor antagonist. In some embodiments, the NMDA receptor antagonist is memantine.

In some embodiments, the methods disclosed herein include wherein the composition is administered intranasal. In some embodiments, the method includes wherein the composition is administered as a dry powder and/or by a nebulizer. In some embodiments, the method includes wherein the composition is administered topically, through injection, and/or orally. In one instance the method includes wherein the composition is administered orally. In another instance the method includes wherein the composition is administered as a lozenge, a powder, a tablet, a gel-cap, a gelatin, a liquid solution, a food, in a food, and/or a dissolvable film.

In some embodiments, the methods disclosed herein include wherein at least one of the biomarker(s) binds to amyloid. In some embodiments, the method includes wherein amyloid aggregation is decreased. In one instance the method includes wherein the biomarkers in the administered composition act synergistically in decreasing amyloid aggregation in comparison to the additive amount of decrease in amyloid aggregation expected for each individual biomarker in the administered composition. In some embodiments, the method includes wherein amyloid secretion is decreased. In one instance the method includes wherein the biomarkers in the administered composition act synergistically in decreasing amyloid secretion in comparison to the additive amount of decrease in amyloid secretion expected for each individual biomarker in the administered composition. In some embodiments, the method includes wherein both soluble and insoluble amyloid levels are decreased.

In some embodiments, the methods disclosed herein include wherein tau level is decreased. In some embodiments, the method includes wherein phosphorylated tau level and/or phosphorylation of tau is decreased.

In some embodiments, the methods disclosed herein includes wherein protein misfolding levels are decreased. In some embodiments, the methods disclosed herein includes wherein protein aggregation levels are decreased.

In some embodiments, the methods disclosed herein includes wherein reactive oxygen species levels and/or free radical levels are decreased.

In some embodiments, the methods disclosed herein include wherein neuro-inflammation is decreased. In some embodiments, the method includes wherein the IL-4 to IL-2 ratio is increased.

In some embodiments, the methods disclosed herein include wherein cognition is increased.

In some embodiments, the methods disclosed herein include wherein uptake of curcumin and/or a functional derivative thereof into a subject is increased when compared to the uptake of curcumin and/or a functional derivative thereof without any of biomarkers 1 through 16, 18, and/or 19.

In some embodiments, the methods disclosed herein include wherein the composition further includes at least one turmerone and has a weight ratio of curcumin and/or functional derivative thereof to turmerones of between 0.5 to 0.9.

In some embodiments, the methods disclosed herein include wherein at least 30% of the curcumin and/or functional derivative thereof present in the composition passes into the serum of the subject. In some embodiments, the method includes wherein at least 10 mg of curcumin and/or functional derivative thereof passes into the serum of the subject. In some embodiments, the method includes wherein the Tₘₐₓ for curcumin and/or functional derivative thereof of is 20 to 120 minutes, 20 to 110 minutes, 30 to 150 minutes, 25 to 100 minutes, or 30 to 90 minutes in the serum of the subject after administration to the subject. In some embodiments, the method includes wherein the Cₘₐₓ for curcumin and/or functional derivative thereof of is at least 5 micromolar, at least 6 micromolar, at least 10 micromolar, or at least 11 micromolar in the serum of the subject after administration to the subject. In some embodiments, the method includes wherein the Tₘₐₓ for biomarker 1 is 5 to 120 minutes, 2 to 100 minutes, 7 to 150 minutes, or 10 to 100 minutes in the serum of the subject after administration to the subject. In some embodiments, the method includes wherein the Tₘₐₓ for biomarker 2 is 2 to 60 minutes, 1 to 45 minutes, 5 to 120 minutes, or 5 to 50 minutes in the serum of the subject after administration to the subject. In some embodiments, the method includes wherein the Tₘₐₓ for biomarker 6 is 10 to 180 minutes, 5 to 150 minutes, 15 to 210 minutes, or 15 to 150 minutes in the serum of a subject after administration to the subject. In some embodiments, the method includes wherein the Tₘₐₓ for biomarker 12 is 5 to 20 minutes, 2 to 15 minutes, 7 to 30 minutes, or 7 to 15 minutes in the serum of a subject after administration to the subject.

Disclosed herein are methods of treating a side effect and/or adverse event associated with a subject taking at least one acetylcholinesterase inhibitor, NMDA receptor antagonist, and/or curcumin. In some embodiments, the method includes administering any one of the compositions disclosed herein to the subject, wherein at least one side effect and/or adverse event associated with a subject taking at least one acetylcholinesterase inhibitor, NMDA receptor antagonist, and/or curcumin is decreased.

Disclosed herein are methods of preventing a side effect and/or adverse event associated with a subject taking at least one acetylcholinesterase inhibitor, NMDA receptor antagonist, and/or curcumin. In some embodiments, the method includes administering any one of the compositions disclosed herein to the subject, wherein at least one side effect and/or adverse event associated with a subject taking at least one acetylcholinesterase inhibitor, NMDA receptor antagonist, and/or curcumin is decreased in comparison to an amount and/or intensity of the at least one side effect and/or adverse event expected if the subject did not take any one of the compositions disclosed herein.

Disclosed herein are methods of increasing curcumin and/or functional derivative thereof uptake into the serum of a subject. In some embodiments, the method includes administering any one of the compositions disclosed herein to the subject, wherein curcumin and/or functional derivative thereof uptake is increased in comparison to administration of curcumin and/or functional derivative thereof without any of biomarkers 1 through 16, 18, or 19. In some embodiments, a disease, disorder, condition, cause of such, and/or symptom of such demonstrated to be treated or prevented by curcumin in in vitro, in vivo, and/or clinical studies is treated or prevented in the subject.

Disclosed herein are methods of increasing curcumin and/or functional derivative thereof uptake into the cerebrospinal fluid of a subject. In some embodiments, the method includes administering any one of the compositions disclosed herein to the subject, wherein curcumin and/or functional derivative thereof uptake is increased in comparison to administration of curcumin and/or functional derivative thereof without any of biomarkers 1 through 16, 18, or 19. In one instance, the method includes wherein the administration of any one of the compositions disclosed herein to the subject provides at least 1 mg of curcumin and/or functional derivative thereof into the cerebrospinal fluid of the subject. In some embodiments, a disease, disorder, condition, cause of such, and/or symptom of such demonstrated to be treated or prevented by curcumin in in vitro, in vivo, and/or clinical studies is treated or prevented in the subject.

Disclosed herein are methods of providing at least one of biomarker(s) 1 through 16, 18, or 19 into the cerebrospinal fluid of a subject. In some embodiments, the method includes administering any one of the compositions disclosed herein to the subject, wherein at least one of the biomarker(s) 1 through 16, 18, or 19 enters the cerebrospinal fluid of the subject.

Disclosed herein are methods of labeling amyloid. In some embodiments, the method includes contacting amyloid with any one of the composition disclosed herein. In some embodiments, the method includes wherein the amyloid labeled is (3 amyloid.

Disclosed herein are methods of labeling tau protein. In some embodiments, the method includes contacting tau with any one of the compositions disclosed herein.

Disclosed herein are methods of producing a composition disclosed herein. In some embodiments, the method includes wherein the method of producing produces a composition having an at least 90%, preferably at least 95% or at least 98% batch-to-batch chemical consistency of relative abundance for the biomarkers.

In some aspects of the invention, the composition may further comprise one or more nutraceutical and/or pharmaceutically acceptable carriers or diluents. These carriers/diluents can be adjuvants, excipients, or vehicles such as preserving agents, fillers, disintegrating agents, wetting agents, emulsifiers, suspending agents, sweeteners, flavorings, fragrance, antibacterial agents, antifungal agents, lubricating agents, vitamins, polymers, siloxane containing compounds, essential oils, structuring agents, and dispensing agents. Each carrier is acceptable in the sense of being compatible with the other ingredients of the formulation and not injurious to the subject. In some aspects of the invention, the carrier can include at least one hydrophilic polymeric compound selected from the group consisting of a gum, a cellulose ether, an acrylic resin, a carbohydrate carrier, talc, lactose, mannitol, glucose, water, gelatin, a protein-derived compound, polyvinyl pyrrolidone, magnesium stearate, and any combination thereof. Non-limiting examples of diluents/carriers are identified throughout this specification and are incorporated into this section by reference. The amounts of such ingredients can range from 0.0001% to 99.9% by weight or volume of the composition, or any integer or range in between as disclosed in other sections of this specification, which are incorporated into this paragraph by reference.

The composition can be stored for one month, 6 months, 12 months, 18 months, or 24 months at room temperature. In some aspects of the invention, the composition is formulated as a powder, a tablet, a gel-cap, a bead, an edible tablet, a food, in a food, a dissolvable film, a liquid capable of being dispersed through the air, a gelatin, a lotion, a transdermal patch, or a liquid solution for oral administration. In some aspects of the invention, the formulated composition can be comprised in a solid nanoparticle, a lipid-containing nanoparticle, a lipid-based carrier, a sealed conduit, a straw, sealed bag, or any combination thereof. In other aspects of the invention, the composition can be formulated for administration by injection.

Kits that include the compositions of the present invention are also contemplated. In certain embodiments, the composition is comprised in a container. The container can be a bottle, dispenser, package, or a straw. The container can dispense a predetermined amount of the composition. In certain aspects, the compositions are dispensed as a pill, a tablet, a capsule, a transdermal patch, an edible chew, a cream, a lotion, a gel, spray, mist, dollop, a powder, or a liquid. The container can include indicia on its surface. The indicia can be a word, an abbreviation, a picture, or a symbol.

It is contemplated that any embodiment discussed in this specification can be implemented with respect to any method or composition of the invention, and vice versa. Furthermore, compositions of the invention can be used to achieve methods of the invention.

Also contemplated is a product that includes the composition of the present invention. In non-limiting aspects, the product can be a nutraceutical product. The nutraceutical product can be those described in other sections of this specification or those known to a person of skill in the art. In other non-limiting aspects, the product can be a pharmaceutical product. The pharmaceutical and/or nutraceutical product can be those described in other sections of this specification or those known to a person of skill in the art. Non-limiting examples of products include a pill, a tablet, an edible chew, a capsule, a cream, a lotion, a gel, a spray, a mist, a dissolving film, a transdermal patch, or a liquid, etc.

Also disclosed are the following Embodiments 1 to 107 of the present invention. Embodiment 1 is a composition comprising: curcumin and/or a functional derivative of curcumin and biomarker 1 having an accurate mass of 120.094 amu and having a relative abundance of at least 2.17%; wherein the biomarker 1 is found in Curcuma longa; and wherein the relative abundance is relative to 25 mg/ml salicylic acid spiked in 0.5 mg/ml of the composition dissolved in ethanol. Embodiment 2 is the composition of Embodiment 1, further comprising any one of, or any combination of, or all of the following additional biomarkers: biomarker 2 having an accurate mass of 134.110 amu and having a relative abundance of at least 0.31%; biomarker 6 having an accurate mass of 200.157 amu and having a relative abundance of at least 0.47%; and biomarker 12 having an accurate mass of 232.146 amu and having a relative abundance of at least 2.38%, wherein the biomarkers are found in. Curcuma longa, and wherein the relative abundance is relative to 25 mg/ml salicylic acid spiked in 0.5 mg/ml of the composition dissolved in ethanol. Embodiment 3 is the composition of Embodiment 2, having at least 2, 3, or 4 of biomarkers 1, 2, 6, and 12. Embodiment 4 is the composition of any one of Embodiments 1 to 3, wherein the composition further comprises one or more of: biomarker 3 having an accurate mass of 150.104 amu and having a concentration of at least 0.04% by weight; biomarker 4 having an accurate mass of 176.120 amu and having a relative abundance of at least 0.96%; biomarker 5 having an accurate mass of 192.091 amu and having a relative abundance of at least 1.74%; biomarker 7 having an accurate mass of 202.172 amu and having a relative abundance of at least 0.87%; biomarker 8 having an accurate mass of 204.188 amu and having a relative abundance of at least 0.30%; biomarker 9 having an accurate mass of 216.151 amu and having a relative abundance of at least 10.75%; biomarker 10 having an accurate mass of 218.203 amu and having a relative abundance of at least 4.00%; biomarker 11 having an accurate mass of 220.183 amu and having a relative abundance of at least 0.72%; biomarker 13 having an accurate mass of 234.162 amu and having a relative abundance of at least 3.52%; biomarker 14 having an accurate mass of 256.240 amu and having a relative abundance of at least 0.25%; biomarker 15 having an accurate mass of 308.105 amu and having a concentration of at least 1.50% by weight; biomarker 16 having an accurate mass of 338.115 amu and having a concentration of at least 1.67% by weight; biomarker 18 having an accurate mass of 372.157 amu and having a concentration of at least 0.88% by weight; and biomarker 19 having an accurate mass of 450.261 amu and having a relative abundance of at least 0.61%, wherein each biomarker is found in Curcuma longa, and wherein the relative abundance is relative to 25 mg/ml salicylic acid spiked in 0.5 mg/ml of the composition dissolved in ethanol. Embodiment 5 is the composition of any of Embodiments 1 to 4, wherein the mass of each biomarker is the mass as determined by a Direct Analysis in Real Time-TOF (DART-TOF) mass spectrometer. Embodiment 6 is the composition of any one of Embodiments 1 to 5, wherein at least one of the biomarker(s) are synthetically obtained. Embodiment 7 is the composition of any one of Embodiments 1 to 6, wherein at least one of the biomarker(s) are isolated from a plant. Embodiment 8 is the composition of Embodiment 7, wherein at least one of the biomarkers(s) are isolated from Curcuma longa. Embodiment 9 is the composition of any one of Embodiments 1 to 8, wherein the composition has an at least. 90%, preferably at least 95%, or at least 98% batch-to-batch chemical consistency of relative abundance for the biomarkers. Embodiment 10 is the composition of any one of Embodiments 1 to 9, wherein the composition further comprises at least one drug. Embodiment 11 is the composition of any one of Embodiments 1 to 10, wherein the composition further comprises at least one acetylcholinesterase inhibitor and/or a N-methyl-D-aspartate (NMDA) receptor antagonist. Embodiment 12 is the composition of Embodiment 11, wherein the at least one acetylcholinesterase inhibitor is donepezil, tacrine, galantamine, rivastigmine, salts thereof or any combination thereof and/or wherein the at least one NMDA receptor antagonist is memantine. Embodiment 13 is the composition of any of Embodiments 1 to 12, wherein the composition further comprises at least one anti-inflammatory drug. Embodiment 14 is the composition of Embodiment 13, wherein the at least one anti-inflammatory drug is a nonsteroidal anti-inflammatory drug. Embodiment 15 is the composition of Embodiment 14, wherein the nonsteroidal anti-inflammatory drug is acetylsalicylic acid, ibuprofen, ketoprofen, or naproxen, salts thereof, or any combination thereof. Embodiment 16 is the composition of any one of Embodiments 1 to 15, wherein the composition is formulated for intranasal administration. Embodiment 17 is the composition of Embodiment 16, wherein the composition is administered as a dry powder and/or by a nebulizer. Embodiment 18 is the composition of any one of Embodiments 1 to 15, wherein the composition is formulated for topical application, administration through injection, and/or oral administration. Embodiment 19 is the composition of Embodiment 18, wherein the composition is formulated for oral administration. Embodiment 20 is the composition of Embodiment 19, wherein the composition is a lozenge, a powder, a tablet, a gel-cap, a gelatin, a liquid solution, a food, in a food, and/or a dissolvable film. Embodiment 21 is the composition of any of Embodiments 1 to 20, wherein at least one of the biomarker(s) is capable of binding to amyloid. Embodiment 22 is the composition of any of Embodiments 1 to 21, wherein at least one of the biomarker(s) is capable of preventing amyloid from aggregating. Embodiment 23 is the composition of any of Embodiments 1 to 22, wherein the composition is formulated to decrease amyloid secretion. Embodiment 24 is the composition of any of Embodiments 1 to 23, wherein the composition is formulated to decrease both soluble and insoluble amyloid levels. Embodiment 25 is the composition of any of Embodiments 1 to 24, wherein the composition is formulated to decrease tau. Embodiment 26 is the composition of any of Embodiments 1 to 25, wherein the composition is formulated to decrease phosphorylated tau and/or phosphorylation of tau. Embodiment 27 is the composition of any of Embodiments 1 to 26, wherein the composition is formulated to decrease neuro-inflammation, protein misfolding, and/or protein degredation. Embodiment 28 is the composition of any of Embodiments 1 to 27, wherein the composition is formulated to increase the ratio of IL-4 to IL-2. Embodiment 29 is the composition of any of Embodiments 1 to 28, wherein the composition is formulated to increase cognition. Embodiment 30 is the composition of any of Embodiments 1 to 29, wherein the composition is formulated to inhibit COX1 and/or COX 2 or a pathway thereof. Embodiment 31 is the composition of any of Embodiments 1 to 30, wherein the composition is formulated to inhibit 5LOX or a pathway thereof. Embodiment 32 is the composition of any of Embodiments 1 to 31, wherein the composition is formulated to have an antioxidant activity. Embodiment 33 is the composition of any of Embodiments 1 to 32, wherein the composition is formulated to scavenge a free radical. Embodiment 34 is the composition of any of Embodiments 1 to 33, wherein the composition is formulated to increase a Th2 response. Embodiment 35 is the composition of any of Embodiments 1 to 34, wherein the composition is formulated to treat and/or prevent a neurological disease, disorder, and/or condition. Embodiment 36 is the composition of Embodiment 35, wherein the composition is formulated to treat and/or prevent a degenerative/protein misfolding disease, disorder, and/or condition, cerebrovascular disease, disorder, and/or condition, inflammatory disease, disorder, and/or condition, trauma/closed head injury, epilepsy, and/or neoplasm. Embodiment 37 is the compositions of Embodiment 35, wherein the composition is formulated to treat and/or prevent Alzheimer's disease, Parkinson's disease, a Lewy body disease, frontotemporal degeneration, progressive supranuclear palsy, amyotrophic lateral sclerosis, multisystem atrophy, cerebral amyloidosis, spinocerebellar atrophy, ischemic stroke, reperfusion injury, cerebral vasospasm, multiple sclerosis, CNS lupus, a concussion, a contusion, chronic traumatic encephalopathy, a generalized seizure disorder, a partial seizure disorder, a metastatic tumor, and/or a primary CNS tumor. Embodiment 38 is the compositions of Embodiment 35, wherein the composition is formulated to treat and/or prevent Alzheimer's disease. Embodiment 39 is the composition 7.7746275.1 of any of Embodiments 1 to 38, wherein the composition is formulated as an anti-nausea. Embodiment 40 is the composition of any of Embodiments 1 to 39, wherein the composition is formulated to treat a side effect and/or adverse event associated with a subject taking at least one acetylcholinesterase inhibitor, NMDA receptor antagonist, and/or curcumin. Embodiment 41 is the composition of any of Embodiments 1 to 39, wherein the composition is formulated to prevent a side effect and/or adverse event associated with a subject taking at least one acetylcholinesterase inhibitor, NMDA receptor antagonist, and/or curcumin. Embodiment 42 is the composition of any of Embodiments 1 to 41, wherein the composition is formulated to increase the uptake of curcumin and/or an analog thereof into a subject when compared to the uptake of curcumin and/or an analog thereof without any of biomarkers 1 through 16, 18, and/or 19. Embodiment 43 is the composition of any of Embodiments 1 to 42, wherein the composition further comprises at least one turmerone and has a weight ratio of curcumin and/or an analog thereof to turmerones of between 0.5 to 0.9. Embodiment 44 is the composition of any of Embodiments 1 to 43, wherein the composition is formulated to provide at least 30% of the curcumin and/or functional derivative thereof present in the composition into the serum of a human administered the composition. Embodiment 45 is the composition of any of Embodiments 1 to 44, wherein the composition is formulated to provide at least 10 mg of curcumin and/or functional derivative thereof into the serum of a human administered the composition. Embodiment 46 is the composition of any of Embodiments 1 to 45, wherein the composition is formulated to provide a Tₘₐₓ for curcumin and/or functional derivative thereof of 20 to 120 minutes in the serum of a human subject after administration to the subject. Embodiment 47 is the composition of any of Embodiments 1 to 46, wherein the composition is formulated to provide a Cₘₐₓ for curcumin and/or functional derivative thereof of at least 5 micromolar in the serum of a human subject after administration to the subject. Embodiment 48 is the composition of any of Embodiments 1 to 47, wherein the composition is formulated to provide a Tₘₐₓ for biomarker 1 of 5 to 120 minutes in the serum of a human subject after administration to the subject. Embodiment 49 is the composition of any of Embodiments 2 to 48, wherein the composition is formulated to provide a Tₘₐₓ for biomarker 2 of 2 to 60 minutes in the serum of a human subject after administration to the subject. Embodiment 50 is the composition of any of Embodiments 2 to 49, wherein the composition is formulated to provide a Tₘₐₓ for biomarker 6 of 10 to 180 minutes in the serum of a human subject after administration to the subject. Embodiment 51 is the composition of any of Embodiments 2 to 50, wherein the composition is formulated to provide a Tₘₐₓ for biomarker 12 of 5 to 20 minutes in the serum of a human subject after administration to the subject. Embodiment 52 is the composition of any of Embodiments 1 to 51, wherein the composition is formulated to provide curcumin and/or functional derivative thereof present in the composition into the cerebrospinal fluid of a human administered the composition. Embodiment 53 is the composition of any of Embodiments 1 to 52, wherein the composition is formulated to provide at least 1 mg of curcumin and/or functional derivative thereof into the cerebrospinal fluid of a human administered the composition. Embodiment 54 is the composition of any of Embodiments 1 to 53, wherein the composition is formulated to provide at least one of the biomarker(s) 1 through 16, 18, or 19 into the cerebrospinal fluid of a human administered the composition. Embodiment 55 is the composition of any of Embodiments 1 to 54, further comprising an imaging agent. Embodiment 56 is the composition of Embodiment 55, wherein the imaging agent is covalently bound to at least one of the biomarker(s) 1 through 16, 18, or 19. Embodiment 57 is the composition of Embodiment 55, wherein the imaging agent is not covalently bound to any of the biomarker(s) 1 through 16, 18, or 19. Embodiment 58 is a method of treating a subject at risk for and/or having a neurological disease, disorder, and/or condition, the method comprising administering any one of the compositions of Embodiments 1 to 57 to the subject, and wherein the neurological disease, disorder, and/or condition is ameliorated in the subject and/or the onset is delayed in comparison to the expected onset of the neurological disease, disorder, and/or condition if the patient had not been treated. Embodiment 59 is the method of Embodiment 58, wherein the neurological disease, disorder, and/or condition is a degenerative/protein misfolding disease, disorder, and/or condition, a cerebrovascular disease, disorder, and/or condition, an inflammatory disease, disorder, and/or condition, a trauma/closed head injury, an epilepsy, and/or a neoplasm. Embodiment 60 is the method of Embodiment 58, wherein the neurological disease, disorder, and/or condition is Alzheimer's disease, Parkinson's disease, a Lewy body disease, frontotemporal degeneration, progressive supranuclear palsy, amyotrophic lateral sclerosis, multisystem atrophy, cerebral amyloidosis, spinocerebellar atrophy, ischemic stroke, reperfusion injury, cerebral vasospasm, multiple sclerosis, CNS lupus, a concussion, a contusion, chronic traumatic encephalopathy, a generalized seizure disorder, a partial seizure disorder, a metastatic tumor, and/or a primary CNS tumor. Embodiment 61 is the method of Embodiment 58, wherein the neurological disease, disorder, and/or condition is Alzheimer's disease. Embodiment 62 is the method of Embodiment 61, wherein the subject is identified as having amyloid secretion, amyloid aggregation, tau hyperphosphorylation, neuro-inflammation, or decreased cognition, or any combination thereof. Embodiment 63 is the method of any one of Embodiments 58 to 62, wherein the subject is administered a total amount of between 1 and 10,000 mg, between 10 and 5,000 mg, between 50 and 2,500 mg, or between 100 and 1,000 mg of the composition during a 24 hour period. Embodiment 64 is the method of any one of Embodiments 58 to 63, wherein at least one of the biomarker(s) 1 through 16, 18, or 19 is synthetically obtained. Embodiment 65 is the method of any one of Embodiments 58 to 64, wherein at least one of the biomarker(s) 1 through 16, 18, or 19 is isolated from plant. Embodiment 66 is the method of Embodiment 65, wherein at least one of the biomarker(s) is isolated from Curcuma longa. Embodiment 67 is the method of any one of Embodiments 58 to 66, wherein the composition has an at least 95% batch-to-batch chemical consistency of relative abundance for the biomarkers. Embodiment 68 is the method of any one of Embodiments 58 to 67, wherein the composition further comprises an a acetylcholinesterase inhibitor and/or a N-methyl-D-aspartate (NMDA) receptor antagonist. Embodiment 69 is the method of Embodiment 68, wherein the acetylcholinesterase inhibitor is donepezil, tacrine, galantamine, rivastigmine, salts thereof, or any combination thereof and/or wherein the at least one NMDA receptor antagonist is memantine. Embodiment 70 is the method of Embodiment 69, wherein the acetylcholinesterase inhibitor is donepezil, a salt thereof, or any combination thereof. Embodiment 71 is the method of any one of Embodiments 58 to 70, wherein the composition is administered intranasal. Embodiment 72 is the method of any one of Embodiments 58 to 71, wherein the composition is administered as a dry powder and/or by a nebulizer. Embodiment 73 is the method of any one of Embodiments 58 to 70, wherein the composition is administered topically, through injection, and/or orally. Embodiment 74 is the method of Embodiment 73, wherein the composition is administered orally. Embodiment 75 is the method of Embodiment 74, wherein the composition is administered as a lozenge, a powder, a tablet, a gel-cap, a gelatin, a liquid solution, a food, in a food, and/or a dissolvable film. Embodiment 76 is the method of any one of Embodiments 61 to 75, wherein at least one of the biomarker(s) binds to amyloid. Embodiment 77 is the method of any of Embodiments 61 to 76, wherein amyloid aggregation is decreased. Embodiment 78 is the method of Embodiment 77, wherein the biomarkers in the administered composition act synergistically in decreasing amyloid aggregation in comparison to the additive amount of decrease in amyloid aggregation expected for each individual biomarker in the administered composition. Embodiment 79 is the method of any of Embodiments 61 to 78, wherein amyloid secretion is decreased. Embodiment 80 is the method of Embodiment 79, wherein the biomarkers in the administered composition act synergistically in decreasing amyloid secretion in comparison to the additive amount of decrease in amyloid secretion expected for each individual biomarker in the administered composition. Embodiment 81 is the method of any of Embodiments 61 to 79, wherein both soluble and insoluble amyloid levels are decreased. Embodiment 82 is the method of any of Embodiments 61 to 81, wherein tau level is decreased. Embodiment 83 is the method of any of Embodiments 61 to 82, wherein phosphorylated tau level and/or phosphorylation of tau is decreased. Embodiment 84 is the method of any of Embodiments 58 to 83, wherein reactive oxygen species levels and/or free radical levels are decreased, protein aggregation is decreased, and/or protein misfolding is decreased. Embodiment 85 is the method of any of Embodiments 58 to 83, wherein neuro-inflammation is decreased. Embodiment 86 is the method of any of Embodiments 58 to 85, wherein the IL-4 to IL-2 ratio is increased. Embodiment 87 is the method of any of Embodiments 58 to 86, wherein cognition is increased. Embodiment 88 is the method of any of Embodiments 58 to 87, wherein uptake of curcumin and/or a functional derivative thereof into a subject is increased when compared to the uptake of curcumin and/or a functional derivative thereof without any of biomarkers 1 through 16, 18, and/or 19. Embodiment 89 is the method of any of Embodiments 58 to 88, wherein the composition further comprises at least one turmerone and has a weight ratio of curcumin and/or functional derivative thereof to turmerones of between 0.5 to 0.9. Embodiment 90 is the method of any of Embodiments 58 to 89, wherein at least 30% of the curcumin and/or functional derivative thereof present in the composition passes into the serum of the subject. Embodiment 91 is the method of any of Embodiments 58 to 90, wherein at least 10 mg of curcumin and/or functional derivative thereof passes into the serum of the subject. Embodiment 92 is the method of any of Embodiments 58 to 91, wherein the Tₘₐₓ for curcumin and/or functional derivative thereof of is 20 to 120 minutes, 20 to 110 minutes, 30 to 150 minutes, 25 to 100 minutes, or 30 to 90 minutes in the serum of the subject after administration to the subject. Embodiment 93 is the method of any of Embodiments 58 to 92, wherein the Cₘₐₓ for curcumin and/or functional derivative thereof of is at least 5 micromolar, at least 6 micromolar, at least 10 micromolar, or at least 11 micromolar in the serum of the subject after administration to the subject. Embodiment 94 is the method of any of Embodiments 58 to 93, wherein the Tₘₐₓ for biomarker 1 is 5 to 120 minutes, 2 to 100 minutes, 7 to 150 minutes, or 10 to 100 minutes in the serum of the subject after administration to the subject. Embodiment 95 is the method of any of Embodiments 58 to 94, wherein the Tₘₐₓ for biomarker 2 is 2 to 60 minutes, 1 to 45 minutes, 5 to 120 minutes, or 5 to 50 minutes in the serum of the subject after administration to the subject. Embodiment 96 is the method of any of Embodiments 58 to 95, wherein the Tₘₐₓ for biomarker 6 is 10 to 180 minutes, 5 to 150 minutes, 15 to 210 minutes, or 15 to 150 minutes in the serum of a subject after administration to the subject. Embodiment 97 is the method of any of Embodiments 58 to 96, wherein the Tₘₐₓ for biomarker 12 is 5 to 20 minutes, 2 to 15 minutes, 7 to 30 minutes, or 7 to 15 minutes in the serum of a subject after administration to the subject. Embodiment 98 is a method of treating a side effect and/or adverse event associated with a subject taking at least one acetylcholinesterase inhibitor, NMDA receptor antagonist, and/or curcumin, the method comprising administering any one of the compositions of Embodiments 1 to 57 to the subject, wherein at least one side effect and/or adverse event associated with a subject taking at least one acetylcholinesterase inhibitor, NMDA receptor antagonist, and/or curcumin is decreased. Embodiment 99 is a method of preventing a side effect and/or adverse event associated with a subject taking at least one acetylcholinesterase inhibitor, NMDA receptor antagonist, and/or curcumin, the method comprising administering any one of the compositions of Embodiments 1 to 57 to the subject, wherein at least one side effect and/or adverse event associated with a subject taking at least one acetylcholinesterase inhibitor, NMDA receptor antagonist, and/or curcumin is decreased in comparison to an amount and/or intensity of the at least one side effect and/or adverse event expected if the subject did not take any one of the compositions of Embodiments 1 to 57. Embodiment 100 is a method of increasing curcumin and/or functional derivative thereof uptake into the serum of a subject, the method comprising administering any one of the compositions of Embodiments 1 to 57 to the subject, wherein curcumin and/or functional derivative thereof uptake is increased in comparison to administration of curcumin and/or functional derivative thereof without any of biomarkers 1 through 16, 18, or 19. Embodiment 101 is a method of increasing curcumin and/or functional derivative thereof uptake into the cerebrospinal fluid of a subject, the method comprising administering any one of the compositions of Embodiments 1 to 57 to the subject, wherein curcumin and/or functional derivative thereof uptake is increased in comparison to administration of curcumin and/or functional derivative thereof without any of biomarkers 1 through 16, 18, or 19. Embodiment 102 is the method of Embodiment 101, wherein the administration of any one of the compositions of Embodiments 1 to 57 to the subject provides at least 1 mg of curcumin and/or functional derivative thereof into the cerebrospinal fluid of the subject. Embodiment 103 is a method of providing at least one of biomarker(s) 1 through 16, 18, or 19 into the cerebrospinal fluid of a subject, the method comprising administering any one of the compositions of Embodiments 1 to 57 to the subject, wherein at least one of the biomarker(s) 1 through 16, 18, or 19 enters the cerebrospinal fluid of the subject. Embodiment 104 is a method of labeling amyloid, the method comprising contacting amyloid with the composition of any of Embodiments 1 to 57. Embodiment 105 is the method of Embodiment 104, wherein the amyloid labeled is p amyloid. Embodiment 106 is a method of labeling tau protein, the method comprising contacting tau with the composition of any of Embodiments 1 to 57. Embodiment 107 is a method of producing a composition of any of Embodiments 1 to 57, wherein the method of producing produces a composition having an at least 90%, preferably at least 95% or at least 98% batch-to-batch chemical consistency of relative abundance for the biomarkers.

“Therapeutic agent” encompasses the compounds specifically claimed herein. It also encompasses such compounds together with nutraceutical and/or pharmaceutically acceptable salts thereof. Useful salts are known to those skilled in the art and include salts with inorganic acids, organic acids, inorganic bases, or organic bases. Therapeutic agents useful in the present invention are those compounds that affect a desired, beneficial, and often pharmacological, effect upon administration to a human or an animal, whether alone or in combination with other nutraceutical and/or pharmaceutical excipients or inert ingredients.

The term “biomarker” refers to the compound defined as the biomarker, analogues thereof, derivatives thereof, salt forms thereof, or salt forms of any analogue or derivative thereof.

The term “accurate mass” refers to a measured mass of a molecule experimentally determined for an ion of known charge. The units for accurate mass include atomic mass units (amu) and milli unified atomic mass units (mmu). The term “molecular weight” refers to the average weight of the molecule with all of the different isotopic compositions present in a compound but weighted for their natural abundance.

The term “relative abundance” refers to the abundance of a compound of interest relative to the abundance of a reference compound. In particular aspects, relative abundance is the raw intensity of a mass spectrometry peak for the compound of interest over the raw intensity of a mass spectrometry peak for a reference compound. In one non-limiting instance, the mass spectrometry peaks can be obtained by the use of DART-TOF mass spectrometry. In another particular aspect, the reference compound is a compound that is spiked, or doped, into a sample containing the compound of interest. In yet another particular aspect, the reference compound is a compound that does not exist in the sample previous to its addition to the sample for determining relative abundance. In another particular aspect, the reference compound can be salicylic acid.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

“Patient,” “subject,” or “individual” refers to a mammal (e.g., human, primate, dog, cat, bovine, ovine, porcine, equine, mouse, rat, hamster, rabbit, or guinea pig). In particular aspects, the patient, subject, or individual is a human.

“Inhibiting” or “reducing” or any variation of these terms includes any measurable decrease or complete inhibition to achieve a desired result.

“Effective” or “treating” or “preventing” or any variation of these terms means adequate to accomplish a desired, expected, or intended result.

“Analogue” and “analog,” when referring to a compound, refers to a modified compound wherein one or more atoms have been substituted by other atoms, or wherein one or more atoms have been deleted from the compound, or wherein one or more atoms have been added to the compound, or any combination of such modifications. Such addition, deletion or substitution of atoms can take place at any point, or multiple points, along the primary structure comprising the compound.

“Derivative,” in relation to a parent compound, refers to a chemically modified parent compound or an analogue thereof, wherein at least one substituent is not present in the parent compound or an analogue thereof. One such non-limiting example is a parent compound which has been covalently modified. Typical modifications are amides, carbohydrates, alkyl groups, acyl groups, esters, pegylations and the like.

A “therapeutically equivalent” compound is one that has essentially the same effect in the treatment of a disease or condition as one or more other compounds. A compound that is therapeutically equivalent may or may not be chemically equivalent, bioequivalent, or generically equivalent.

“Parenteral injection” refers to the administration of small molecule drugs via injection under or through one or more layers of skin or mucus membranes of an animal, such as a human.

“Bioavailability” refers to the extent to which the therapeutic agent is absorbed from the formulation.

“Pharmaceutically acceptable carrier” refers to a pharmaceutically acceptable solvent, suspending agent or vehicle for delivering a composition or drug compound of the present invention to a mammal such as an animal or human.

“Nutraceutical acceptable carrier” refers to a nutraceutical acceptable solvent, suspending agent or vehicle for delivering a compound of the present invention to an animal such as a mammal or human.

“Pharmaceutically acceptable” ingredient, excipient or component is one that is suitable for use with humans and/or animals without undue adverse side effects (such as toxicity, irritation and allergic response) commensurate with a reasonable benefit/risk ratio.

“Nutraceutical acceptable” ingredient, excipient or component is one that is suitable for use with humans and/or animals without undue adverse side effects (such as toxicity, irritation and allergic response) commensurate with a reasonable benefit/risk ratio.

The term “about” or “approximately” or “substantially unchanged” are defined as being close to as understood by one of ordinary skill in the art, and in one non-limiting embodiment the terms are defined to be within 10%, preferably within 5%, more preferably within 1%, and most preferably within 0.5%. Further, “substantially non-aqueous” refers to less than 5%, 4%, 3%, 2%, 1%, or less by weight or volume of water.

The use of the word “a” or “an” when used in conjunction with the term “comprising” in the claims and/or the specification may mean “one,” but it is also consistent with the meaning of “one or more,” “at least one,” and “one or more than one.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. With respect to the transitional phase “consisting essentially of,” in one non-limiting aspect, a basic and novel characteristic of the compositions and methods disclosed in this specification includes the compositions' abilities to reduce or prevent Alzheimer's disease and/or related causes and/or symptoms, such as, but not limited to inflammation, protein misfolding, and/or protein aggregation.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 Detection of curcumin and biomarkers 1, 2, 6, and 12 in human blood serum from an oral dose of HSRx-888.

FIG. 2 Detection of curcumin in human blood serum (average from 5 subjects) from an oral dose of 50 mg of HSRx-888 (containing 35 mg curcumin).

FIG. 3 Detection of biomarker 1 in human blood serum from an oral dose of HSRx-888 (average from 5 human subjects).

FIG. 4 Detection of biomarker 2 in human blood serum from an oral dose of HSRx-888 (average from 5 human subjects).

FIG. 5 Detection of biomarker 6 in human blood serum from an oral dose of HSRx-888 (average from 5 human subjects).

FIG. 6 Detection of biomarker 12 in human blood serum from an oral dose of HSRx-888 (average from 5 human subjects).

FIG. 7 HSRx-888 (HSG0888) demonstrates a dose dependent inhibition of β-amyloid aggregation. HSRx-888 effectively inhibits Aβ₁₋₄₂ at micromolar (μM) concentrations. Aβ aggregation assays were conducted with the synthetic Aβ₁₋₄₂ peptide incubated with HSRx-888, HSG0838, HSG0848 or single-molecule standards (Curcumin (Cur), demethoxycurcumin (DMC), bisdemethoxycurcumin (BDMC), and tetrahydrocurcumin (THC)) at varying concentrations from 0 to 30 μg/mL. Aggregation was measured 5 days after a single treatment event by the thioflavin T method.

FIG. 8 HSRx-888 (HSG0888) significantly decreases β-amyloid generation in a concentration-dependent manner. HSRx-888 significantly reduced Aβ generation both Aβ₁₋₄₀ and Aβ₁₋₄₂ peptides in SweAPP N2a cells in a concentration-dependent manner. SweAPP N2a cells were treated with a concentration range of 3-30 μg/mL with each compound for 12 hours and the Aβ_{1-40,42} peptides were analyzed in conditioned media from SweAPP N2a cells by ELISA.

FIGS. 9 A and B HSRx-888 (HSS-888) reduces cerebral amyloidosis in Tg2576 mice. HSRx-888 was orally administered to 8 month old Tg2576 mice. HSRx-888 treatment significantly reduced Aβ deposition in these mice compared to both control and THC treatments. A—staining of cingulate cortex and entorhinal cortex of Aβ depositions. B—plaque burden in mean % with standard error in entorhinal cortex (EC), hippocampus (H), and cingulate cortex (CC).

FIGS. 10 A and B HSRx-888 (HSS-888) reduces both soluble and insoluble β-amyloid levels in Tg2576 mouse brain homogenates. Mouse brain homogenates were analyzed for Aβ levels by ELISA. Orally administered HSRx-888 significantly reduced soluble and insoluble forms of Aβ_{1-40,42} compared to soluble and insoluble controls (A and B, respectively).

FIGS. 11 A and B HSRx-888 (HSS-888) decreases phosphorylated tau protein in Tg2576 mice. Mice treated with HSRx-888 showed an 80% decrease in pTau relative to control mice. Anterior quarter brain homogenates from the treated mice were analyzed by Western blot analysis. **=p<0.01.

FIGS. 12 A and B HSRx-888 (HSS-888) enhances Th2 cellular immune responses. HSRx-888 treatment increased cytokines IL-2 and IL-4 indicating HSRx-888 affords microglia protection via the anti-inflammatory activity of specific cytokines. Primary cultures of microglia were established from the mice following sacrifice and stimulated for 24 h with anti-CD 3 antibody. Data were represented as pg of each cytokine per total intracellular protein (mg). **=p<0.01.

FIGS. 13 A and B Proposed, non-binding mechanism of action for reduction of β-amyloid aggregation. Model of the interaction between βA(1-42) monomers and biomarker 15 (BDMC). Strong intermolecular interactions occur between Tyr₁₀ and biomarker 1 which allows biomarker 15 to surround His₁₃ and His₁₄ effectively preventing Phe₁₉ and Phe₂₀ from binding and forming oligomers (A). Biomarker 15 can also bind to Gly₃₃, Met₃₅, and Gly₃₇ disrupting the stabilizing intermolecular interactions of the PA(1-42) oligomers (B).

FIG. 14 Dose-dependent inhibition of 2-diphenyl-1-picrylhydrazyl Radical Assay (DPPH) by HSRx-888. The HSRx-888 IC₅₀ value is 19.2 μg mL⁻¹ (R²=0.731, N=10).

FIGS. 15 A, B and C Dose-dependent inhibition of COX1 (A), COX2 (B), and 5LOX (C) by HSRx888. The HSRx-888 IC₅₀ values are 100.6 μg mL⁻¹ (R²=0.907, N=36), 23.0 μg mL⁻¹ (R²=0.874, N=24), and 256.3 μg mL⁻¹ (R²=0.999, N=8), respectively.

### DETAILED DESCRIPTION

The inventors have surprisingly found that a combination of several compounds that can be found in turmeric can prevent and treat Alzheimer's disease, inflammation, protein misfolding, and protein aggregation. The inventors have also found that specific relative concentrations of the compounds act to enhance the ability of the combined compounds to prevent and treat Alzheimer's disease, inflammation, protein misfolding, and protein aggregation. In addition, the inventors have found that using compounds of the present invention with additional dugs enhance the ability of the combined compounds to prevent and treat Alzheimer's disease, inflammation, protein misfolding, and protein aggregation. Without wishing to be bound by theory, it is believed that the compounds and compositions disclosed herein may be effective through the capability to increase curcumin uptake into a subject, including a human subject's blood plasma and cerebrospinal fluid, the composition's anti-inflammatory capacity, the ability of the composition to bind amyloid, the ability of the composition to decrease amyloid aggregation, and the ability of the composition to decrease amyloidosis.

The compounds and compositions disclosed herein are capable of treating, ameliorating, and preventing the symptoms associated with Alzheimer's disease and inflammation and side effects associated with the taking of drugs to treat Alzheimer's disease and inflammation, such as nausea. Non-limiting examples of symptoms and/or causes of Alzheimer's disease include amyloid aggregation, increased amyloid secretion, increased amyloid production, neuritic plaques, loss of normal physiological functions of amyloid, hyperphosphorylation of tau, increased neurofibrillary tangles, increased toxic species of tau, increased levels of tau, neuro-inflammation, etc. Additional non-limiting examples of symptoms of Alzheimer's disease include decreased cognition, memory impairment, confusion, visual impairment, impairment of spatial recognition, reduced vocabulary, depression, changes in mood, etc.

The compounds and compositions disclosed herein are capable of reducing protein aggregation and protein misfolding, providing benefits in treating and/or preventing neurodegenerative disorders such as Alzheimer's disease (beta-amyloid and phosphorylated tau proteins), Parkinson's disease (alpha-synuclein protein), Dementia with Lewy bodies (beta-amyloid, phosphorylated tau and alpha-synuclein proteins), Frontotemporal dementias (tau protein), Spongiform encephalopathies (prion protein), as well as in many other central and systemic amyloidosis.

Further, the combinations disclosed herein provide benefits in treatment and/or prevention of other neurological diseases, disorders, and/or conditions such as, but not limited to, degenerative/protein misfolding disorders, cerebrovascular diseases, inflammatory diseases, trauma/closed head injuries, epilepsies, and/or neoplasms. Non-limiting examples of degenerative/protein misfolding diseases, disorders, and/or conditions include Alzheimer's, Parkinson's, Lewy body, frontotemporal degeneration, progressive supranuclear palsy, amyotrophic lateral sclerosis, multisystem atrophy, cerebral amyloidosis, spinocerebellar atrophy. Non-limiting examples of cerebrovascular diseases, disorders, and/or conditions include ischemic stroke, reperfusion injury, and cerebral vasospasm. Non-limiting examples of inflammatory diseases, disorders, and/or conditions include multiple sclerosis and CNS lupus. Non-limiting examples of trauma/closed head injuries include concussions, contusions, and chronic traumatic encephalopathy. Non-limiting examples of epilepsies include generalized seizure disorders and partial seizure disorders. Non-limiting examples of neoplasms include metastatic and primary CNS tumors.

### A. Compounds of the Composition

The composition of the present invention can include curcumin (368.126 amu) and one or more of the biomarkers found in Curcuma longa (turmeric) defined by accurate mass of 120.094 amu (Biomarker 1), 134.110 amu (Biomarker 2), 150.104 amu (Biomarker 3), 176.120 amu (Biomarker 4), 192.091 amu (Biomarker 5), 200.157 amu (Biomarker 6), 202.172 amu (Biomarker 7), 204.188 amu (Biomarker 8), 216.151 amu (Biomarker 9), 218.203 amu (Biomarker 10), 220.183 amu (Biomarker 11), 232.146 amu (Biomarker 12), 234.162 amu (Biomarker 13), 256.240 amu (Biomarker 14), 308.105 amu (Biomarker 15), 338.115 amu (Biomarker 16), 372.157 amu (Biomarker 18), and 450.261 amu (Biomarker 19), and combinations thereof. Without wishing to be bound by theory, it is believed that the biomarkers increase the uptake of curcumin into the serum of a subject and/or the cerebrospinal fluid of a subject, binds amyloid, decreases protein aggregation, decreases protein misfiling, and decrease inflammation.

In a particular embodiment, the biomarker or combination of biomarkers has a 90% batch-to-batch chemical consistency of relative abundance for the biomarkers. In another particular embodiment, the compound or combination of compounds has a 95% and/or 98% batch-to-batch chemical consistency of relative abundance for the biomarkers.

In some aspects of the invention, the compounds of the composition and derivatives and analogues can be made through known synthetic methods. In some aspects of the invention, the compounds of the composition and/or the composition can be synthetically obtained by producing the compound(s) and/or the compositions according to methods known to one of skill in the art in chemical synthesis. In some aspects, the compound(s) and/or the compositions are synthesized through organic chemistry methods.

In some aspects of the invention, the compounds of the composition and/or the composition can be isolated from extracts of an organism such as fruits, plants, animals, fungi, bacteria, and/or archaea. Non-limiting examples of plants include Curcuma longa. The compounds of the composition or the composition can be extracted from the organism using known extraction methods, such as contacting the extract with CO₂ at 40-80° C. and 80-900 bar, or contacting the extract with H₂O or any combination of EtOH:H₂O, and separating the extract with any method utilizing polymer separation. A non-limiting example of a polymer used for polymer separation includes ADS 5 polymer (Nankai University, China). The extract can include curcumin and any one or combination of compounds defined by accurate mass of 120.094 amu (Biomarker 1), 134.110 amu (Biomarker 2), 150.104 amu (Biomarker 3), 176.120 amu (Biomarker 4), 192.091 amu (Biomarker 5), 200.157 amu (Biomarker 6), 202.172 amu (Biomarker 7), 204.188 amu (Biomarker 8), 216.151 amu (Biomarker 9), 218.203 amu (Biomarker 10), 220.183 amu (Biomarker 11), 232.146 amu (Biomarker 12), 234.162 amu (Biomarker 13), 256.240 amu (Biomarker 14), 308.105 amu (Biomarker 15), 338.115 amu (Biomarker 16), 372.157 amu (Biomarker 18), and 450.261 amu (Biomarker 19) that are found in Curcuma longa.

In some aspects of the invention, one or more of the compounds of the composition and derivatives and analogues thereof can be made through known synthetic methods known by one of skill in the art and one or more of the compounds of the composition and derivatives and analogues thereof may be isolated from other sources, such as, but not limited to, extracts of fruits and plants.

### B. Actives Defined by DART TOF/MS

The accurate mass and relative abundances described herein are based on experiments using particular instruments and particular settings and can change from instrument to instrument. There is variability in each measurement. Thus, the accurate mass and relative abundances arc defined as being close to as understood by one of ordinary skill in the art. In one non-limiting embodiment the terms are defined to be within 30%, preferably within 20%, preferably 10%, preferably within 5%, more preferably within 1%, and most preferably within 0.5%. In one non-limiting embodiment, the accurate mass has an error of within +/−20 mmu, preferably 10 mmu, more preferably within 5 mmu, and most preferably within 1 mmu. In one non-limiting embodiment, the relative abundance has an error of +/−20%, preferably 10%, preferably within 5%, and more preferably within 1%, and most preferably within 0.5%.

In a non-limiting example, the compounds of the present invention can be identified using Direct Analysis in Real Time (DART) Time of Flight/Mass Spectrometry (TOF/MS). Specifically, a JEOL DART™ AccuTOF-mass spectrometer from Jeol USA of Peabody, MA. (JMS-T100LC) can be used. Accurate mass can be determined by subtracting the mass of a proton (1.007825 amu) from the measured mass of the ions produced from the sample. The mass of compounds may be determined in a sample by directly introducing the sample to the ion stream by means of a Dip-IT sampler and a Dip-IT sampler holder (ionSense™). While no sample preparation is required for a simple analysis with the DART, a chemical doped/spiked solution can be used for quantitation relative to a known quantity. As a non-limiting example, the reference compound is not present in the sample until added to serve as a reference and can therefore be used to create a quantitative chemical profile of the bioactive molecules. The settings for the DART ion source can be the following:
Gas: He
Flow: 2.52 LPM @ 50 PSI
Temperature: 250 C
Needle Voltage: 3000V
Grid Electrode Voltage: 250V
Discharge Electrode Voltage: 400V
   The settings for the JEOL AccuTOF MS can be the following:
Peaks Voltage: 1000V
Orifice 1 Temperature: 120 C
Detector Voltage: 2600V
Reflectron Voltage: 990.0V

Samples can be analyzed in six replicates by DART-TOF MS. These six replicates can be analyzed to create a single, averaged, filtered, and statistically significant DART fingerprint of the sample. This processed fingerprint can then be used to determine the presence of the bioactive markers by comparison of masses. Due to the initial discovery and identification of these bioactive markers, a simple mass comparison is sufficient to determine their presence in any extract or mixture of chemicals.

All MS have a mass tolerance—a range of acceptable reported masses surrounding the predicted [M+H] or [M−H] value. For the AccuTOF, that mass tolerance is less than 20 millimass units (mmu) (predicted mass+/−10 mmu). Given the same sample and ion source, other TOF-MS may have a higher or lower mass tolerance.

In another non-limiting example, the compounds of the present invention can be determined by DART TOF/MS by using a JEOL DART™ AccuTOF-mass spectrometer from Jeol USA of Peabody, MA. (JMS-T100LC) executed in the positive ion mode ([M+H]⁺) using the following settings for the DART ion source:
Gas: He
Flow: 3.98 L/min
Needle voltage: 3500 V
Temperature: 300° C.
Electrode 1 Voltage: 150 V
Electrode 2 Voltage: 250 V,
   The settings for the JEOL AccuTOF MS can be the following:
Peaks Voltage: 1000V
Orifice 1 Voltage: 20 V
Ring Lens Voltage: 5 V
Orifice 2 Voltage: 5 V
Detector Voltage: 2550V

Calibrations can be performed internally with each sample using a 10% (weight/volume) solution of PEG 600 from Ultra Chemical of North Kingston, R.I. that provided mass markers throughout the required mass range of 100-1000 amu. Calibration tolerances can be held to 5 mmu. Samples can be introduced into the DART He plasma using the closed end of a borosilicate glass melting point capillary tube until a signal is achieved in the total-ion chromatogram (TIC). The next sample can then be introduced when the TIC returned baseline levels.

### C. Agents to Treat or Prevent Alzheimer's Disease or Symptoms Thereof

It is contemplated that the compositions of the present invention can include agents to treat or prevent Alzheimer's disease or symptoms thereof. Such agents are compounds or compositions that are used to decrease the symptoms or causes of Alzheimer's disease. Non-limiting examples of symptoms or causes of Alzheimer's disease include amyloid aggregation, increased amyloid secretion, increased amyloid production, neuritic plaques, loss of normal physiological functions of amyloid, hyperphosphorylation of tau, increased neurofibrillary tangles, increased toxic species of tau, increased levels of tau, neuro-inflammation, etc. Additional non-limiting examples of symptoms of Alzheimer's disease include decreased cognition, memory impairment, confusion, visual impairment, impairment of spatial recognition, reduced vocabulary, depression, changes in mood, etc.

Non-limiting examples of agents to treat or prevent Alzheimer's disease or symptoms thereof include acetylcholinesterase inhibitors, NMDA receptor antagonist, and/or curcumin. Acetylcholinesterase inhibitors are used to inhibit acetylcholinesterase enzyme. Acetylcholinesterase enzyme breaks down the neurotransmitter acetylcholine. Non-limiting examples of acetylcholinesterase inhibitors include donepezil, tacrine, galantamine, and rivastigmine. Non-limiting examples of NMDA receptor antagonist include memantine. Some acetylcholinesterase inhibitors have side effects such as nausea. Administration of large amounts of curcumin may also cause gastrointestinal problems, including nausea. In one embodiment, the compositions disclosed herein further include at least one acetylcholinesterase inhibitors, which may be, but is not limited to, donepezil, tacrine, galantamine, and rivastigmine. In some embodiments, the composition is formulated to decrease the side effects of acetylcholinesterase inhibitors and/or curcumin, which may be, but is not limited to nausea. In one embodiment, the compositions disclosed herein further include at least one NMDA receptor antagonist, which may be, but is not limited to, memantine.

### D. Anti-Inflammatory Agents

It is contemplated that the compositions of the present invention can include anti-inflammatory agents. Anti-Inflammatory agents are compounds or compositions that are used to decrease the inflammatory response in a subject or decrease the effects of an inflammatory response. Non-limiting examples of anti-inflammatory agents include corticosteroids and nonsteroidal anti-inflammatory drugs. Non-limiting examples of nonsteroidal anti-inflammatory drugs include acetylsalicylic acid, ibuprofen, ketoprofen, and naproxen. Some anti-inflammatory drugs inhibit COX1 or COX2, or a pathway thereof. Some anti-inflammatory drugs inhibit 5LOX or the 5LOX pathway. Some anti-inflammatory agents increase anti-inflammatory cytokines such as IL-2 and IL-4. Some anti-inflammatory agents reduce a Th1 response and/or increase a Th2 response. In some embodiments, the compositions disclosed herein further include at least one additional anti-inflammatory agent, which may be, but is not limited to acetylsalicylic acid, ibuprofen, ketoprofen, and naproxen.

### E. Amounts of Ingredients

It is contemplated that the compositions of the present invention can include any amount of the ingredients discussed in this specification. The compositions can also include any number of combinations of additional ingredients described throughout this specification (e.g., stabilizers, fillers, pharmaceutically and/or nutraceutical acceptable salts, and/or additional pharmaceutical and/or nutraceutical ingredients). The concentrations of the any ingredient within the compositions can vary. In non-limiting embodiments, for example, the compositions can comprise, consisting essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight or volume of the total composition or relative abundance. A person of ordinary skill in the art would understand that the concentrations can vary depending on the addition, substitution, and/or subtraction of ingredients in a given composition.

### F. Additional Components

The compound of the present invention can be formulated into any suitable composition form for administration to a human or non-human animal patient.

The composition may consist of the claimed compounds alone or may include the compounds and any suitable additional component, such as one or more pharmaceutically and/or nutraceutical acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Each carrier must be acceptable in the sense of being compatible with the other ingredients of the formulation and not injurious to the patient.

### 1. Excipients

Excipients employed in the compositions of the present invention can be solids, semi-solids, liquids or combinations thereof. Preferably, the excipients are solids. Compositions of the invention containing excipients can be prepared by any known technique that comprises, for example, admixing an excipient with the claimed compounds. A pharmaceutical composition of the invention contains a desired amount of the claimed compounds per dose unit and, if intended for oral administration, can be in the form, for example, of a tablet, a caplet, a pill, a hard or soft capsule, a lozenge, a cachet, a dispensable powder, granules, a suspension, an elixir, a dispersion, or any other form reasonably adapted for such administration. If intended for intranasal administration, it can be in the form, for example, a dry powder, a nebulizer, or any other form reasonably adapted for such administration. If intended for parenteral administration, it can be in the form, for example, of a suspension or transdermal patch. If intended for rectal administration, it can be in the form, for example, of a suppository. Presently particular are oral dosage forms that are discrete dose units each containing a predetermined amount of the claimed compounds such as tablets or capsules.

### 2. Carriers/Diluents

Suitable carriers or diluents Illustratively include, but are not limited to, either individually or in combination, lactose, including anhydrous lactose and lactose monohydrate; starches, including directly compressible starch and hydrolyzed starches (e.g., Celutab™ and Emdex™), mannitol, sorbitol, xylitol, dextrose (e.g., Cerelose™ 2000) and dextrose monohydrate, dibasic calcium phosphate dihydrate, sucrose-based diluents, confectioner's sugar, monobasic calcium sulfate monohydrate, calcium sulfate dihydrate, granular calcium lactate trihydrate, dextrates, inositol, hydrolyzed cereal solids, amylose, celluloses including microcrystalline cellulose, food grade sources of alpha- and amorphous cellulose (e.g., RexcelJ), powdered cellulose, hydroxypropylcellulose (HPC) and hydroxypropylmethylcellulose (HPMC), calcium carbonate, glycine, clay, bentonite, block co-polymers, polyvinylpyrrolidone, and the like. Such carriers or diluents, if present, constitute in total about 5% to about 99.999%, about 10% to about 85%, and 20% to about 80%, of the total weight of the composition. The carrier, carriers, diluent, or diluents selected preferably exhibit suitable flow properties and, where tablets are desired, compressibility.

### 3. Disintegrant

Compositions of the invention optionally can include one or more pharmaceutically and/or nutraceutical acceptable disintegrants as excipients, particularly for tablet formulations. Suitable disintegrants include, but are not limited to, either individually or in combination, starches, including sodium starch glycolate and pregelatinized corn starches, clays, celluloses such as purified cellulose, microcrystalline cellulose, methylcellulose, carboxymethylcellulose and sodium carboxymethylcellulose, croscarmellose sodium, alginates, crospovidone, and gums such as agar, guar, locust bean, karaya, pectin and tragacanth gums. Disintegrants may be added at any suitable step during the preparation of the composition, particularly prior to granulation or during a lubrication step prior to compression. Such disintegrants, if present, constitute in total preferably about 0.2% to about 30%, preferably about 0.2% to about 10%, and more preferably about 0.2% to about 5%, of the total weight of the composition.

### 4. Binders

The compositions of the present invention can include binding agents or adhesives particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Such binding agents may also prevent or inhibit crystallization or recrystallization of a co-crystal of the present invention once the salt has been dissolved in a solution. Suitable binding agents and adhesives include, but are not limited to, either individually or in combination, acacia; tragacanth, sucrose, gelatin, glucose, starches such as, but not limited to, pregelatinized starches, celluloses such as, but not limited to, methylcellulose and carmellose sodium, alginic acid and salts of alginic acid; magnesium aluminum silicate, PEG, guar gum, polysaccharide acids, bentonites, povidone, polymethacrylates, hydroxypropylcellulose, and ethylcellulose. Such binding agents and/or adhesives, if present, constitute in total preferably about 0.5% to about 25%, preferably about 0.75% to about 15%, and more preferably about 1% to about 10%, of the total weight of the pharmaceutical composition. Many of the binding agents are polymers comprising amide, ester, ether, alcohol or ketone groups and, as such, can be included in pharmaceutical compositions of the present invention. Polyvinylpyrrolidones is an non-limiting example of a binder used for slow release tablets. Polymeric binding agents can have varying molecular weight, degrees of crosslinking, and grades of polymer. Polymeric binding agents can also be copolymers, such as block co-polymers that contain mixtures of ethylene oxide and propylene oxide units. Variation in these units' ratios in a given polymer may affect properties and performance.

### 5. Wetting Agents

Wetting agents can be used in the compositions of the present invention. Wetting agent can be selected to maintain the crystal in close association with water, a condition that may improve bioavailability of the composition. Such wetting agents can also be useful in solubilizing or increasing the solubility of crystals. Surfactants can be used as wetting agents. Non-limiting examples of surfactants that can be used as wetting agents in compositions of the invention include quaternary ammonium compounds, for example benzalkonium chloride, benzethonium chloride and cetylpyridinium chloride, dioctyl sodium sulfosuccinate, polyoxyethylene alkylphenyl ethers, poloxamers (polyoxyethylene and polyoxypropylene block copolymers), polyoxyethylene fatty acid glycerides and oils, for example polyoxyethylene (8) caprylic/capric mono- and diglycerides, polyoxyethylene (35) castor oil and polyoxyethylene (40) hydrogenated castor oil, polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80, propylene glycol fatty acid esters, for example propylene glycol laurate, sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total preferably about 0.25% to about 15%, preferably about 0.4% to about 10%, and more preferably about 0.5% to about 5%, of the total weight of the pharmaceutical composition.

### 6. Lubricants

Lubricants can be included in the compositions of the present invention. Suitable lubricants include, but are not limited to, either individually or in combination, glyceryl behenate, stearic acid and salts thereof, including magnesium, calcium and sodium stearates; hydrogenated vegetable oils, colloidal silica, talc, waxes, boric acid, sodium benzoate, sodium acetate, sodium fumarate, sodium chloride, DL-leucine, PEG (e.g., Carbowax™ 4000 and Carbowax™ 6000 of the Dow Chemical Company), sodium oleate, sodium lauryl sulfate, and magnesium lauryl sulfate. Such lubricants, if present, constitute in total preferably about 0.1% to about 10%, preferably about 0.2% to about 8%, and more preferably about 0.25% to about 5%, of the total weight of the composition.

### 7. Other Agents

Surfactant, emulsifier, or effervescent agents can be used in the compositions. Emulsifying agents can be used to help solubilize the ingredients within a soft gelatin capsule. Non-limiting examples of the surfactant, emulsifier, or effervescent agent include D-sorbitol, ethanol, carrageenan, carboxyvinyl polymer, carmellose sodium, guar gum, glycerol, glycerol fatty acid ester, cholesterol, white beeswax, dioctyl sodium sulfosuccinate, sucrose fatty acid ester, stearyl alcohol, stearic acid, polyoxyl 40 stearate, sorbitan sesquioleate, cetanol, gelatin, sorbitan fatty acid ester, talc, sorbitan trioleate, paraffin, potato starch, hydroxypropyl cellulose, propylene glycol, propylene glycol fatty acid ester, pectin, polyoxyethylene (105) polyoxypropylene (5) glycol, polyoxyethylene (160) polyoxypropylene (30) glycol, polyoxyethylene hydrogenated castor oil, polyoxyethylene hydrogenated castor oil 40, polyoxyethylene hydrogenated castor oil 60, polyoxyl 35 castor oil, polysorbate 20, polysorbate 60, polysorbate 80, macrogol 400, octyldodecyl myristate, methyl cellulose, sorbitan monooleate, glycerol monostearate, sorbitan monopalmitate, sorbitan monolaurate, lauryl dimethylamine oxide solution, sodium lauryl sulfate, lauromacrogol, dry sodium carbonate, tartaric acid, sodium hydroxide, purified soybean lecithin, soybean lecithin, potassium carbonate, sodium hydrogen carbonate, medium-chain triglyceride, citric anhydride, cotton seed oil-soybean oil mixture, and liquid paraffin.

### G. Vehicles

Various delivery systems are known in the art and can be used to administer a therapeutic agent or composition of the invention, e.g., encapsulation in liposomes, microparticles, microcapsules, receptor-mediated endocytosis and the like. Methods of administration include, but are not limited to, parenteral, intra-arterial, intramuscular, intravenous, intranasal, and oral routes. The compositions can be provided in the form of tablets, lozenges, granules, capsules, pills, ampoule, suppositories or aerosol form. The compositions can also be provided in the form of suspensions, solutions, and emulsions of the active ingredient in aqueous or non-aqueous diluents, syrups, granulates or powders.

### H. Formulation and Administration

The composition may, for example, be a pharmaceutical composition (medicament), and over the counter composition (OTC), a nutraceutical, etc. Compositions according to the present invention include formulations suitable for nasal, oral, or parenteral routes. Non-limiting examples of specific routes include intradermal, subcutaneous, intramuscular, intravenous, local injection, rectal, intranasal inhalation, insufflation, topical (including transdermal, buccal and sublingual), vaginal, parenteral (including subcutaneous, intramuscular, intravenous and intradermal) and pulmonary administration. The formulations can conveniently be presented in unit dosage form and can be prepared by any methods well known in the art. Such methods include the step of bringing into association the active ingredient (or ingredients) with the carrier which constitutes one or more accessory ingredients. In general, the formulations are prepared by uniformly and intimately bringing into association the active ingredient with a suitable carrier, such as liquid carriers or finely divided solid carriers or both, and then if necessary shaping the product. Formulations of the subject invention suitable for oral administration can be presented as discrete units such as capsules, cachets or tablets, each containing a predetermined amount of the active ingredient, or as an oil-in-water liquid emulsion, water-in-oil liquid emulsion, or as a supplement within an aqueous solution, for example, a tea. The active ingredient can also be presented as bolus, electuary, or paste. Useful injectable preparations include sterile suspensions, solutions or emulsions of the compound compositions in aqueous or oily vehicles. The compositions can also contain formulating agents, such as suspending, stabilizing and/or dispersing agent. The formulations for injection can be presented in unit dosage form, e.g., in ampoules or in multidose containers, and can contain added preservatives. Alternatively, the injectable formulation can be provided in powder form for reconstitution with a suitable vehicle, including but not limited to sterile pyrogen free water, buffer, dextrose solution, etc., before use. To this end, the compound compositions can be dried by any art-known technique, such as lyophilization, and reconstituted prior to use.

Formulations suitable for topical administration in the mouth include lozenges comprising the active ingredient in a flavored basis, usually sucrose and acacia or tragacanth, pastilles that include the active ingredient in an inert basis such as gelatin and glycerin, or sucrose and acacia, mouthwashes that include the active ingredient in a suitable liquid carrier, and chocolate comprising the active ingredients.

Formulations suitable for topical administration according to the subject invention can be formulated as an ointment, cream, suspension, lotion, powder, solution, paste, gel, spray, aerosol or oil. Alternatively, a formulation can comprise a patch or a dressing such as a bandage or adhesive plaster impregnated with active ingredients, and optionally one or more excipients or diluents. Topical formulations preferably comprise compounds that facilitate absorption of the active ingredients through the skin and into the bloodstream.

Formulations suitable for intranasal administration, wherein the carrier is a solid, include a coarse powder having a particle size, for example, in the range of about 20 to about 500 microns, which is administered in the manner in which snuff is taken, i.e., by rapid inhalation through the nasal passage from a container of the powder held close up to the nose. Suitable formulations wherein the carrier is a liquid for intranasal administration, such as by the non-limiting examples of a nebulizer, include aqueous or oily solutions of the agent. Formulations preferably can include compounds that facilitate absorption of the active ingredients through the skin and into the bloodstream.

Formulations suitable for parenteral administration include aqueous and non-aqueous isotonic sterile injection solutions which can contain antioxidants, buffers, bacteriostats and solutes which render the formulation isotonic with the blood of the intended recipient; and aqueous and non-aqueous sterile suspensions which can include suspending agents and thickening agents, and liposomes or other microparticulate systems which are designed to target the compound to blood components or one or more organs. The formulations can be presented in unit-dose or multi-dose or multi-dose sealed containers, such as for example, ampoules and vials, and can be stored in a freeze-dried (lyophilized) condition requiring only the addition of the sterile liquid carrier, for example, water for injections, immediately prior to use. Extemporaneous injection solutions and suspensions can be prepared from sterile powders, granules and tablets of the kind previously described.

Liquid preparations for oral administration can take the form of, for example, elixirs, solutions, syrups or suspensions, or they can be presented as a dry product for constitution with water or other suitable vehicle before use. Such liquid preparations can be prepared by conventional means with pharmaceutically and/or nutraceutical acceptable additives such as suspending agents (e.g., sorbitol syrup, cellulose derivatives. or hydrogenated edible fats); emulsifying agents (e.g., lecithin or acacia); non aqueous vehicles (e.g., almond oil, oily esters, ethyl alcohol, or fractionated vegetable oils); and preservatives (e.g., methyl or propyl p hydroxybenzoates or sorbic acid). The preparations can also contain buffer salts, preservatives, flavoring, coloring and sweetening agents as appropriate.

For buccal administration, the compositions can take the form of the non-limiting examples of tablets or lozenges formulated in a conventional manner.

For rectal and vaginal routes of administration, the compound compositions can be formulated as solutions (for retention enemas) suppositories or ointments containing conventional suppository bases such as cocoa butter or other glycerides.

For nasal administration or administration by inhalation or insufflation, the compound compositions can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit can be determined by providing a valve to deliver a metered amount. Capsules and cartridges for use in an inhaler or insufflator (for example capsules and cartridges comprised of gelatin) can be formulated containing a powder mix of the compound and a suitable powder base such as lactose or starch.

For prolonged delivery, the compound compositions can be formulated as a depot preparation for administration by implantation or intramuscular injection. The compound compositions can be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch, which slowly releases the compound compositions for percutaneous absorption, can be used. To this end, permeation enhancers can be used to facilitate transdermal penetration of the compound compositions. Suitable transdermal patches are described in for example, U.S. Pat. Nos. 5,407,713; 5,352,456; 5,332,213; 5,336,168; 5,290,561; 5,254,346; 5,164,189; 5,163,899; 5,088,977; 5,087,240; 5,008,110; and 4,921,475.

Alternatively, other delivery systems can be employed. Liposomes and emulsions are well-known examples of delivery vehicles that can be used to deliver the compound compositions. Certain organic solvents such as dimethylsulfoxide (DMSO) can also be employed, although usually at the cost of greater toxicity.

It should be understood that in addition to the ingredients particularly mentioned above, the formulations useful in the present invention can include other agents conventional in the art regarding the type of formulation in question. For example, formulations suitable for oral administration can include such further agents as sweeteners, thickeners, and flavoring agents. It also is intended that the agents, compositions, and methods of this invention be combined with other suitable compositions and therapies.

In one embodiment, the pharmaceutical and/or nutraceutical compositions of the invention can be administered locally to the area in need of treatment; such local administration can be achieved, for example, by local infusion, by injection, or by means of a catheter. In another embodiment, a compound or composition of the invention is administered in a manner so as to achieve peak concentrations of the active compound at sites of the disease. Peak concentrations at disease sites can be achieved, for example, by intravenously injecting of the agent, optionally in saline, or orally administering, for example, a tablet, capsule, or syrup containing the active ingredient.

### I. Other Pharmaceutical and/or Nutraceutical Agents

Pharmaceutical, OTC, and/or nutraceutical formulations of the invention can be administered simultaneously or sequentially with other drugs or biologically active agents. Examples include, but are not limited to, antioxidants, free radical scavenging agents, analgesics, anesthetics, anorectals, antihistamines, anti-inflammatory agents including non-steroidal anti-inflammatory drugs, antibiotics, antifungals, antivirals, antimicrobials, anti-cancer actives, antineoplastics, biologically active proteins and peptides, enzymes, hemostatics, steroids including hormones and corticosteroids, etc.

### J. Therapeutic Methods And Dosage

Particular unit dosage formulations are those containing a daily dose or unit, daily subdose, or an appropriate fraction thereof, of an agent. Therapeutic amounts can be empirically determined and will vary with the pathology being treated, the subject being treated, and the efficacy and toxicity of the agent. Similarly, suitable dosage formulations and methods of administering the agents can be readily determined by those of ordinary skill in the art.

In some embodiments, a therapeutic method of the present invention can include treating a disease, condition, or disorder by administering to a subject having such disease or condition a stable formulation as described herein in an amount effective to treat the disease, condition, or disorder. In some embodiments, the subject is administered a stable formulation comprising the compounds claimed herein. The disease, condition, or disorder can be Alzheimer's disease, inflammation, protein misfolding and protein aggregation diseases or conditions, and/or a disease with similar symptoms and related diseases, conditions, and disorders. For prophylactic administration, the composition can be administered to a patient at risk of developing one of the previously described conditions.

The amount of composition administered will depend upon a variety of factors, including, for example, the particular indication being treated, the mode of administration, whether the desired benefit is prophylactic or therapeutic, the severity of the indication being treated and the age and weight of the patient, etc. Determination of an effective dosage is well within the capabilities of those skilled in the art. In some aspects of the invention, total dosage amounts of a compound composition will typically be in the range of from about 0.0001 or 0.001 or 0.01 mg/kg of patient/day to about 100 mg/kg patient/day, but may be higher or lower, depending upon, among other factors, the activity of the components, its bioavailability, the mode of administration and various factors discussed above. Dosage amount and interval can be adjusted individually to provide plasma levels of the compound(s) that are sufficient to maintain therapeutic or prophylactic effect. For example, the compounds can be administered once per week, several times per week (e.g., every other day), once per day or multiple times per day, depending upon, among other things, the mode of administration, the specific indication being treated and the judgment of the prescribing physician. Skilled artisans will be able to optimize effective local dosages without undue experimentation.

K. Kits

In another aspect of the present invention, kits for treating a disease, condition or disorder are described herein. For instance, compositions of the present invention can be included in a kit. A kit can include a container. Containers can include a bottle, a metal tube, a laminate tube, a plastic tube, a dispenser, a straw, a pressurized container, a barrier container, a package, a compartment, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a predetermined amount of the composition. In other embodiments, the container can be squeezed (e.g., metal, laminate, or plastic tube) to dispense a desired amount of the composition. The composition can be dispensed as a spray, an aerosol, a liquid, a fluid, a semi-solid, or a solid. In a particular embodiment, the composition is dispensed as a tablet or lozenge. The containers can have spray, pump, or squeeze mechanisms. A kit can also include instructions for employing the kit components as well the use of any other compositions included in the container. Instructions can include an explanation of how to apply, use, and maintain the compositions. The compositions can, if desired, be presented in a pack or dispenser device, which can contain one or more unit dosage forms containing the compound compositions. The pack can, for example, comprise metal or plastic foil, such as a blister pack. The pack or dispenser device can be accompanied by instructions for administration.

### EXAMPLES

The present invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Example 1

### Characterization of Compounds by Accurate Mass, Relative Abundance, and Percent Weight

The inventors have surprisingly found that a combination of several compounds can prevent and treat Alzheimer's disease, protein aggregation, protein misfolding, and inflammation. The inventors have also found that specific relative concentrations of the compounds act to enhance the ability of the combined compounds to prevent and treat these diseases. The compounds of the present invention include curcumin and biomarker compounds defined by compounds found in Curcuma longa with an accurate mass of 120.094 amu (Biomarker 1), 134.110 amu (Biomarker 2), 150.104 amu (Biomarker 3), 176.120 amu (Biomarker 4), 192.091 amu (Biomarker 5), 200.157 amu (Biomarker 6), 202.172 amu (Biomarker 7), 204.188 amu (Biomarker 8), 216.151 amu (Biomarker 9), 218.203 amu (Biomarker 10), 220.183 amu (Biomarker 11), 232.146 amu (Biomarker 12), 234.162 amu (Biomarker 13), 256.240 amu (Biomarker 14), 308.105 amu (Biomarker 15), 338.115 amu (Biomarker 16), 372.157 amu (Biomarker 18), and 450.261 amu (Biomarker 19). These compounds may be produced synthetically or isolated from an organism such as, but not limited to, Curcuma longa. The compounds may be characterized by methods known by one of skill in the art.

Accurate mass and relative abundances described herein are based on experiments using particular instruments and particular settings and can change from instrument to instrument. There is variability in each measurement. Thus, the accurate mass and relative abundances are defined as being “close to” as understood by one of ordinary skill in the art.

Methods for Accurate mass: The compounds were characterized and relative abundance was determined using Direct Analysis in Real Time (DART) ion source combined with Time of Flight/Mass Spectrometry (TOF-MS). Specifically, the DART TOF-MS was a JEOL DART™ AccuTOF-mass spectrometer from Jeol USA of Peabody, MA. (JMS-T100LC). The mass of the compounds were determined in a Curcuma longa extract sample by directly introducing the sample to the ion stream by means of a Dip-IT sampler and a Dip-IT sampler holder (ionSense™)

The settings for the DART ion source were the following:
Gas: He
Flow: 2.52 LPM @ 50 PSI
Temperature: 250 C
Needle Voltage: 3000V
Grid Electrode Voltage: 250V
Discharge Electrode Voltage: 400V

The settings for the JEOL AccuTOF MS were the following:
Peaks Voltage: 1000V
Orifice 1 Temperature: 120 C
Detector Voltage: 2600V
Reflectron Voltage: 990.0V

Extract samples were analyzed in six replicates by DART-TOF MS. These six replicates were analyzed to create a single, averaged, filtered, and statistically significant DART fingerprint of the extract. This processed fingerprint was then used to determine the presence of the bioactive markers by comparison of masses. Due to the initial discovery and identification of these bioactive markers, a simple mass comparison was sufficient to determine their presence in any extract or mixture of chemicals. For the AccuTOF, that mass tolerance is less than 20 millimass units (mmu) (predicted mass+/−10 mmu). Given the same extract and ion source, other TOF mass spectrometers may have a higher or lower mass tolerance.

Methods for Relative Abundance: While no sample preparation is required for a simple analysis with the DART, a salicylic acid doped/spiked solution was used for determining relative abundance of test compositions through quantitation relative to a known quantity. Standards that are well known and that exist naturally in turmeric, such as curcumin, would vary given any number of influences—growing conditions, harvest time, plant health, etc. For purposes of quantifying the biomarkers, the natural variations of curcumin (or other naturally occurring standards) make it unacceptable to use as a basis for an absolute quantification of the biomarkers. In order to remove that inconsistency, a compound that is not native to turmeric (in this case, salicylic acid) was used as the basis for a quantitative chemical profile of the bioactive molecules.

For determining relative abundance of samples with unknown concentrations of the biomarkers disclosed herein, 0.5 mg/ml samples of the disclosed compositions in ethanol were doped/spiked 25 mg/ml salicylic acid. Samples were then analyzed by the DART-TOF method used above.

Method for Determining Percent Weight: Percent weight was determined using the DART-TOF method used for relative abundance; however, salicylic acid was replaced with an available standard for the biomarker.

Table 1 discloses the percent weight and relative abundance of the biomarkers disclosed herein found in non-limiting, particular embodiments of compositions comprising biomarkers 1-16, 18, 19, and curcumin (biomarker 17).

TABLE 1
Percent weight determined by use of a standard and relative abundance
of the biomarkers in particular active compositions determined using
0.5 mg/ml of the compositions spiked with 25 mg/ml salicylic acid.

| | | Minimum | Maximum | | |
|---|---|---|---|---|---|
| | | Relative | Relative | Minimum | Maximum |
| | Accurate | Abundance to | Abundance to | Percent | Percent |
| | Mass | Salicylic Acid | Salicylic Acid | Weight | Weight |
| | (amu) | (−30%) | (+30%) | (−30%) | (+30%) |
| Biomarker 1 | 120.094 | 2.17 | 4.04 | | |
| Biomarker 2 | 134.110 | 0.31 | 0.57 | | |
| Biomarker 3 | 150.104 | | | 0.04 | 0.08 |
| (carvacrol) | | | | | |
| Biomarker 4 | 176.120 | 0.96 | 1.78 | | |
| Biomarker 5 | 192.091 | 1.74 | 3.23 | | |
| Biomarker 6 | 200.157 | 0.47 | 0.88 | | |
| Biomarker 7 | 202.172 | 0.87 | 1.62 | | |
| Biomarker 8 | 204.188 | 0.30 | 0.56 | | |
| Biomarker 9 | 216.151 | 10.75 | 19.96 | | |
| Biomarker 10 | 218.203 | 4.00 | 7.44 | | |
| Biomarker 11 | 220.183 | 0.72 | 1.33 | | |
| Biomarker 12 | 232.146 | 2.38 | 4.41 | | |
| Biomarker 13 | 234.162 | 3.52 | 6.54 | | |
| Biomarker 14 | 256.240 | 0.25 | 0.46 | | |
| Biomarker 15 | 308.105 | | | 1.50 | 2.79 |
| (BDMC) | | | | | |
| Biomarker 16 | 338.115 | | | 1.67 | 3.10 |
| (DMC) | | | | | |
| Biomarker 17 | 368.126 | | | 38.00 | 71.00 |
| (curcumin) | | | | | |
| Biomarker 18 | 372.157 | | | 0.88 | 1.64 |
| (THC) | | | | | |
| Biomarker 19 | 450.261 | 0.61 | 1.13 | | |

### Example 2

### Formulations for Examples 3 Through 8

HSRx-888, a particular embodiment of the disclosed composition that comprises a dose-reliable, turmeric extract comprising 55% by weight curcumin and biomarkers 1 through 16, 18, and 19 with 0.06% biomarker 3, 2.15% biomarker 15, 2.39% biomarker 16, and 1.26% biomarker 18 by weight and relative abundances of 3.11% for biomarker 1, 0.44% for biomarker 2, 1.37% for biomarker 4, 2.49% for biomarker 5, 0.68% for biomarker 6, 1.24% for biomarker 7, 0.43% for biomarker 8, 15.35% for biomarker 9, 5.72% for biomarker 10, 1.02% for biomarker 11, 3.39% for biomarker 12, 5.03% for biomarker 13, 0.35% for biomarker 14, and 0.87% for biomarker 19 and with in vitro and in vivo activity against the causes and symptoms of Alzheimer's disease, protein misfolding, protein aggregation, and inflammation was produced in general according to the methods described in Shytle et al. 2009 and Shytle et al. 2012.

Generally, turmeric (Curcuma longa) was ground and extracted with CO₂ at 40-80° C. and 80-900 bar and polymer separated using ADS 5 polymer (Nankai University, China). The collected fraction may be dried at 50° C. overnight to yield a crystalline powder. The procedure was repeated multiple times to ensure reproducibility of the extract.

### Example 3

### Blood Serum PK and Tolerance Study in Human Subjects

This example concerns data obtained from a study which examined the blood serum pharmacokinetics (PK) of the formulation of Example 2 in normal human volunteer subjects. 50 mg of the formulation was orally administered to volunteer human subjects. The 50 mg dose contained 35 mg curcumin. Blood was drawn and tested at t=0, 5, 10, 20, 30, 40, 60, 120, 180, 240, and 480 minutes after oral administration to 5 human volunteers. The intensity of the peaks for curcumin and/or curcumin and biomarkers 1, 2, 6, and 12 in the blood plasma was determined by DART ToF-MS.

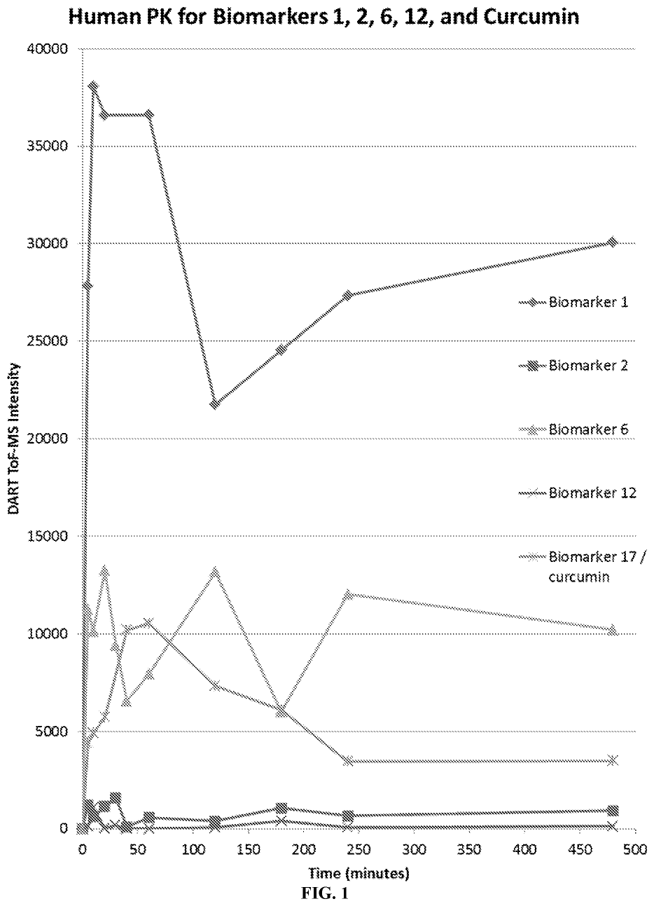

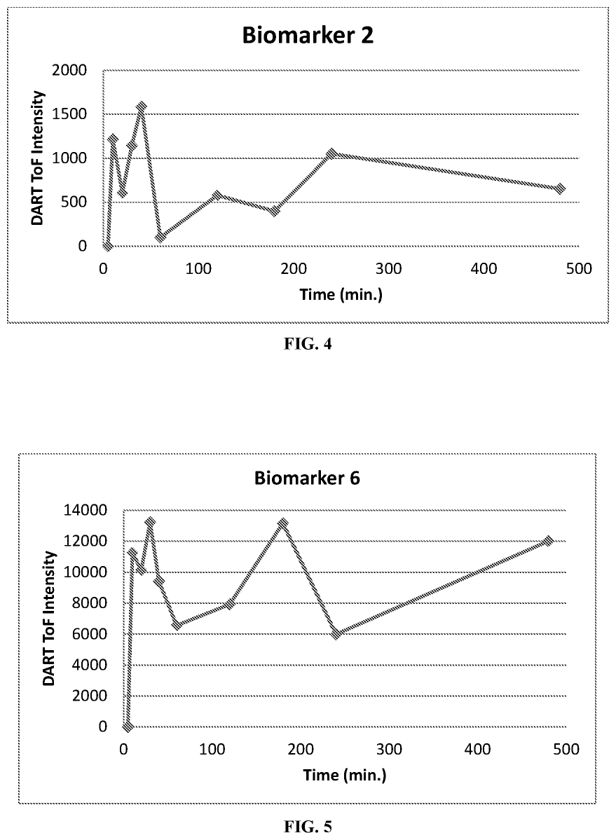

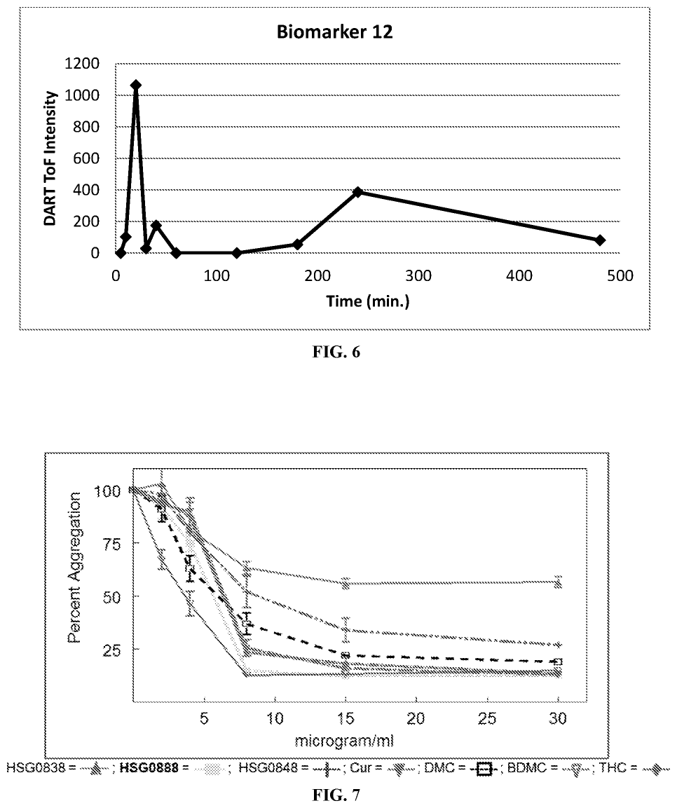

The peaks at each time point were plotted to determine the maximum concentration (Cₘₐₓ) of curcumin and time of maximum concentration (Tₘₐₓ) of curcumin and each biomarker 1, 2, 6, and 12 (FIGS. 1-6). Cₘₐₓ and Tₘₐₓ were determined empirically using the average peak intensity at each time point.

Results: HSRx-888 is an effective inhibitor of Aβ₁₋₄₂ aggregation in vitro as compared to other turmeric extracts such as HSG0838 and HSG0848 (FIG. 7). Further, the results show that HSRx-888 inhibits aggregation to a greater or similar extent to the individual biomarkers found in HSRx-888 (curcumin, DMC, BCMC, and THC) when the individual biomarkers are used at the same dosage as the entire HSRx-888 composition (e.g. 15 micrograms/ml HSRx-888 compared to 15 micrograms curcumin). However, these individual biomarkers are found in HSRx-888 at much lower concentrations (see Table 1), strongly suggesting that the biomarkers in HSRx-888 are acting synergistically. Further, it is expected from this data and additional data disclosed herein that the compositions disclosed herein possess anti-protein aggregation and anti-protein misfolding properties that would be beneficial in treating and/or preventing neurodegenerative disorders such as Alzheimer's disease (beta-amyloid and phosphorylated tau proteins), Parkinson's disease (alpha-synuclein protein), Dementia with Lewy bodies (beta-amyloid, phosphorylated tau and alpha-synuclein proteins), Frontotemporal dementias (tau protein), Spongiform encephalopathies (prion protein), as well as many other central and systemic amyloidosis.

### Example 5

### INHIBITION OF AMYLOID GENERATION In Vivo

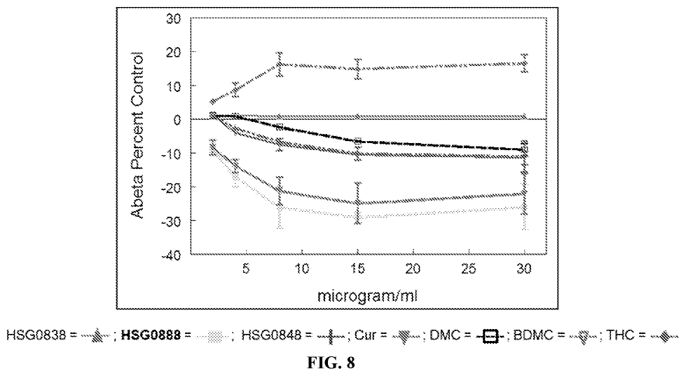

As shown in Shytle et al., 2009, HSRx-888 (HSG0888) significantly reduced Aβ generation for both Aβ₁₋₄₀ and Aβ₁₋₄₂ peptides in SweAPP N2a cells in a concentration-dependent manner (FIG. 8). As explained in Shytle et al., 2009, SweAPP N2a cells were treated with a concentration range of 3-30 μg/mL with each compound for 12 hours and the Aβ_{1-40,42} peptides were analyzed in conditioned media from SweAPP N2a cells by ELISA.

Methodology; “Conditioned media were collected and analyzed at a 1:1 dilution using the method as previously described (Tan et al., 2002) and values were reported as percentage of Aβ₁₋₄₂ secreted relative to control (conditioned medium from untreated N2a SweAPP cells). Quantification of total Aβ species was performed according to published methods (Marambaud et al., 2005; Obregon et al. 2006). Briefly, 6E10 (capture antibody) was coated at 2 μg/mL in phosphate buffered saline (PBS; pH 7.4) into 96-well immunoassay plates overnight at 4° C. The plates were washed with 0.05% (v/v) Tween-20 in PBS five times and blocked with blocking buffer (PBS with 1% BSA, 5% [v/v] horse serum) for 2 h at room temperature.

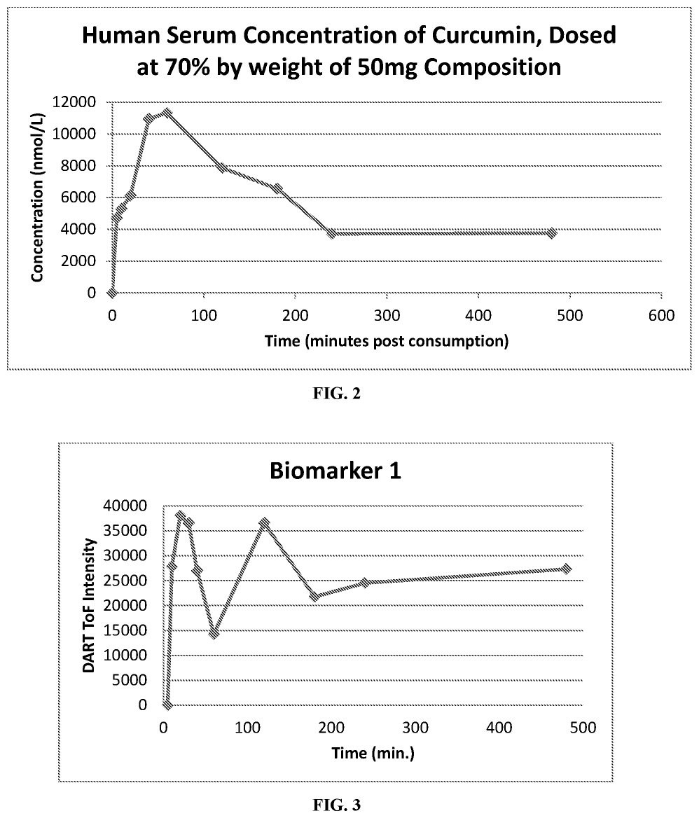

It was determined that a single oral dose of HSRx-888 of 50 mg produced micromolar levels of free, unmodified curcumin in blood (FIG. 2, with a Cₘₐₓ of 11.3 micromolar). Tₘₐₓ for curcumin was shown to be approximately between 40 and 120 minutes. Tₘₐₓ for biomarkers 1, 2, 6, and 12 were approximately 5 to 120 for biomarker 1, 5 and 60 for biomarker 2, 5 and 240 for biomarker 6, and 1 and 30 for biomarker 12. Further, administration of HSRx-888 was found to be well-tolerated.

### Example 4

### Inhibition of Amyloid Aggregation In Vitro

As shown in Shytle et al., 2009, HSRx-888 (HSG0888) demonstrates a dose dependent inhibition of 3-Amyloid (Aβ) aggregation at micromolar concentrations in vitro. (FIG. 7). Aβ aggregation assays were conducted with synthetic Aβ₁₋₄₂ peptide incubated with HSRx-888, other proprietary turmeric extracts (HSG0838, HSG0848) or single-molecule standards (curcumin (Cur), 15% demethoxycurcumin (DMC), 5% bisdemethoxycurcumin (BDMC), and tetrahydrocurcumin (THC)) at varying concentrations from 0 to 30 μg/mL. Aggregation was measured 5 days after a single treatment event by the thioflavin T method as described in Shytle et al., 2009. The thioflavin T method detects mainly mature 3-pleated sheet amyloid fibers.

Methodology: “The presence of Aβ₁₋₄₂ fibers was monitored in solution by thioflavin T fluorescence as described previously (Moore et al., 2004; LeVine, 1993). Briefly, triplicate 15 μL samples of Aβ₁₋₄₂ (25 μM, 95 μg/mL) in 50 mM Tris-HCl buffer (pH 7.4) were removed after incubation of the peptide solution in the presence or absence of optimized turmeric extracts ([HSG0888, HSG0838, HSG0848]) or the curcuminoid standards (Cur, DMC, BDCM, and THC) at concentrations from 0 to 30 μg/mL for up to 120 hours at 37° C. These peptide solutions were each added to 100 μL of 10 μM Thioflavin T in 50 mM glycine/NaOH buffer (pH 9.0) in a black-walled 96-well plate, incubated for 30 minutes at 25° C. before the characteristic change in fluorescence was monitored (Ex 450 nm and Em 482 nm) following binding of thioflavin T to the amyloid fibers by using a Molecular Devices SPECTRAmax GEMINI plate reader. Triplicate samples were scanned three times before and immediately after the addition of the peptide solutions. Results show the mean value of the triplicate samples±the difference between those mean values.” Shytle et al., 2009.

Conditioned medium or Aβ standards were added to the plates and incubated overnight at 4° C. Following 3 washes, biotinylated antibody, 4G8 (0.5 μg/mL in PBS with 1% [w/v] BSA) was added to the plates and incubated for 2 h at room temperature. After 5 washes, streptavidin-horseradish peroxidase (1:200 dilutions in PBS with 1% BSA) was added to the 96-wells for 30 min at room temperature.

Tetramethylbenzidine (TMB) substrate was added to the plates and incubated for 15 minutes at room temperature. A 50 μL aliquot of stop solution (2 N N2SO4) was added to each well of the plates to top the reaction. The optical density of each well was determined immediately on a microplate reader at O.D. 450 nm. The Aβ levels were expressed as a percentage of control (conditioned medium from untreated N2a SweAPP cells).” Shytle et al., 2009.

Results: Untreated SweAPP N2a cells excreted 128 pg total of Aβ₁₋₄₀ and Aβ₁₋₄₂ peptides. HSRx-888 significantly reduced the amount of Aβ₁₋₄₀ and Aβ₁₋₄₂ peptides secreted in a concentration dependent manner. (FIG. 8). The other turmeric extracts showed no to little inhibition. Curcumin also showed significant inhibition. (FIG. 8). Further, the results show that HSRx-888 inhibits secretion to a greater or similar extent to the individual biomarkers found in HSRx-888 (curcumin, DMC, BCMC, and THC) when the individual biomarkers are used at the same dosage as the entire HSRx-888 composition (e.g. 15 micrograms/ml HSRx-888 compared to 15 micrograms curcumin). However, these individual biomarkers are found in HSRx-888 at much lower concentrations (see Table 1), strongly suggesting that the biomarkers in HSRx-888 are acting synergistically.

### Example 6

### Reduction of Cerebral Amyloidosis in Tg2576 Mice

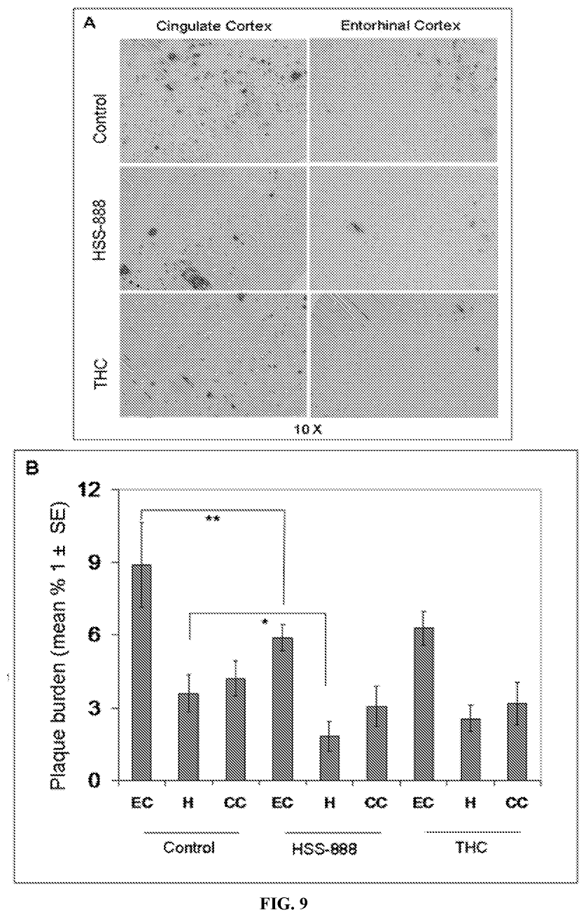

As shown in Shytle et al., 2012, HSRx-888 (HSS-888) reduces cerebral amyloidosis in Tg2576 mice. (FIGS. 9 A and B). As explained in Shytle et al., HSRx-888 was or ally administered to 8 month old Tg2576 mice and Aβ deposition in these mice were analyzed through staining of brain coronal frozen sections with rabbit-polyclonal anti-human Aβ antibody (FIG. 9 A) and quantified using quantitative image analysis (FIG. 9 B).

Methodology: “In Vivo Treatments—Beginning at 8 months of age, Tg2576 treatment mice were administered the optimized turmeric extract [HSRx-888] (0.1% w/w) or THC (0.1% w/w) in NIH31 chow or NIH3I chow alone (Control) for 6 months [n=20 (JO female/10 male)]. All mice were sacrificed at 14 months of age for analyses of Aβ levels and AP load in the brain according to previously described methods (Garcia-Alloza et al., 2006).

Immunohistochemistry—Mice were anesthetized with isofluorane and transcardially perfused with ice-cold physiological saline containing heparin (10 U/mL). Brains were rapidly isolated and quartered using a mouse brain slicer (Muromachi Kikai Co., Tokyo, Japan). The first and second anterior quarters were homogenized for ELISA and Western blot analysis as described below, and the third and fourth posterior quarters were used for microtome or cryostat sectioning. Brains were then fixed in 4% (w/v) paraformaldehyde in PBS at 4° C. overnight and routinely processed in paraffin in a core facility at the Department of Pathology (USF College of Medicine). Five serial coronal sections (5 μm) spaced ˜150 μm apart from each brain section were selected for immunohistochemical staining and image analysis. Sections were routinely deparaffinized and hydrated in a graded series of USP ethanol prior to pre-blocking for 30 min at ambient temperature with serum-free protein block (Dakocytomation, Glostrup, Denmark). The AP immunohistochemical staining was performed using anti-human P-antibody (clone 4G8, 1:100) in conjunction with the VectaStain Elite™ ABC kit coupled with diaminobenzidine substrate. The 4 GB-positive AP deposits were examined under bright-field using an Olympus BX-51 microscope. Quantitative image analysis (conventional “Aβ burden” analysis) was routinely performed for 4G8 immuno-histochemistry. Data are reported as percentage of immunolabeled area captured (positive pixels) divided by the full area captured (total pixels).

Image Analysis—Quantitative image analysis (conventional “Aβ burden” analysis) was performed using stereo logical methods for 4G8 immuno-histochemistry and Congo red histochemistry for brains from Tg2576 mice orally administrated THC, HSRx-888, or NIH31 control chow. Images were obtained using an Olympus BX-51 microscope and digitized using an attached MAGNAFIRE™ imaging system (Olympus, Tokyo, Japan). Briefly, images from five serial sections (5 μm) spaced ˜150 μm apart through each anatomic region of interest (hippocampus or cortical areas) were captured and a threshold optical density was obtained that discriminated staining form background. Manual editing of each field was used to eliminate artifacts. Data are reported as percentage of immunolabeled area captured (positive pixels) divided by the full area captured (total pixels). Quantitative image analysis was performed by a single examiner blinded to sample identities.” Shytle et al., 2012.

Results: HSRx-888 reduced cerebral amyloidosis in Tg2576 mice as shown in FIG. 9 A—staining of cingulate cortex and entorhinal cortex of Aβ depositions and FIG. 9 B-plaque burden in mean % with standard error in entorhinal cortex (EC), hippocampus (H), and cingulate cortex (CC). It is expected from this data and other data provided herein, that the compositions disclosed herein possess anti-protein aggregation and anti-protein misfolding properties that would be beneficial in treating and/or preventing neurodegenerative disorders such as Alzheimer's disease (beta-amyloid and phosphorylated tau proteins), Parkinson's disease (alpha-synuclein protein), Dementia with Lewy bodies (beta-amyloid, phosphorylated tau and alpha-synuclein proteins), Frontotemporal dementias (tau protein), Spongiform encephalopathies (prion protein), as well as many other central and systemic amyloidosis.

### Example 7

### Reduction of Soluble and Insoluble Amyloid Levels in Tg2576 Mice

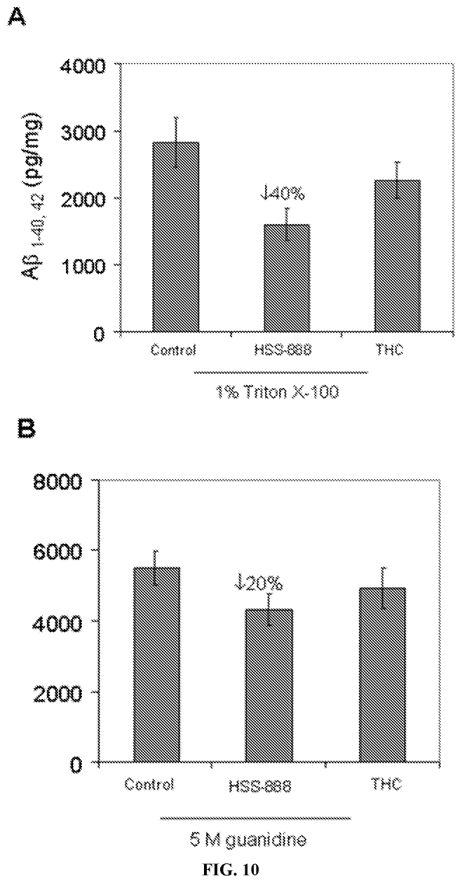

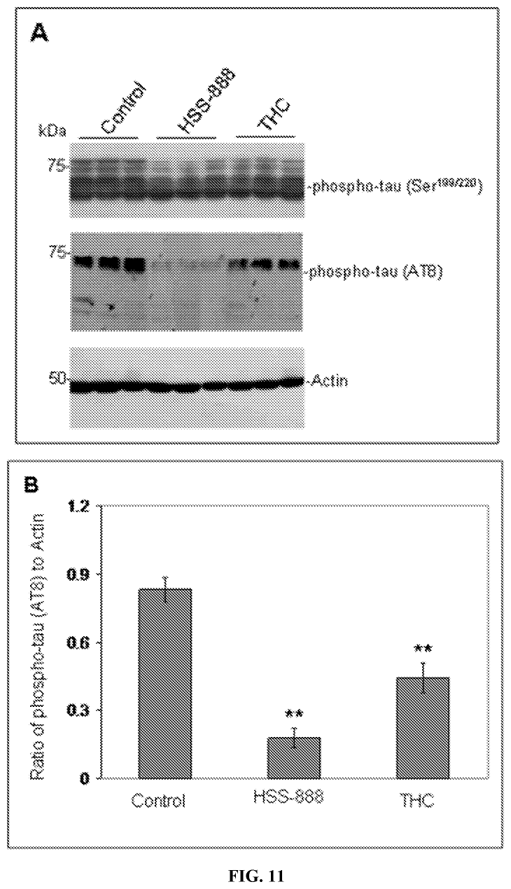

As shown in Shytle et al., 2012, HSRx-888 (HSS-888) reduces both soluble and insoluble β-Amyloid levels in Tg2576 mouse brain homogenates. (FIGS. 10 A and B). Mouse brain homogenates were analyzed for Aβ levels by ELISA. Orally administered HSRx-888 significantly reduced soluble and insoluble forms of Aβ_{1-40,42} compared to soluble and insoluble controls (A and B, respectively).

Methodology: “Mouse brains were isolated under sterile conditions on ice and placed in ice-cold lysis buffer (20 mM Tris, pH 7.5, 150 mM NaCl, 1 mM EDTA, 1 mM EGTA, 1% [v/v] Triton X-100, 2.5 mM sodium pyrophosphate, 1 mM β-glycerolphosphate, 1 mM Na₃VO₄, 1 μg/mL leupeptin, 1 mM PMSF) as previously described (Johnson-Wood et al. 1997). Brains were then sonicated on ice for approximately 3 min, allowed to stand for 15 min at 4° C., and centrifuged at 15,000 rpm for 15 min. Insoluble Aβ_{1-40,42} species were detected by acid extraction of brain homogenates in 5 M guanidine buffer (Rezai-Zadeh et al. 2008), followed by a 1:10 dilution in lysis buffer. Soluble Aβ_{1-40,42} were directly detected in brain homogenates prepared with lysis buffer described above by a 1:10 dilution. Protein levels of homogenate samples were all normalized by BCA protein assay prior to dilution. Aβ_{1-40,42} was quantified using an Immuno-Biological Laboratories non-discriminate Aβ ELISA kit in accordance with the manufacturer's instructions, except that standards included 0.5 M guanidine buffer in some cases.” Shytle et al., 2012.

Results: Orally administered HSRx-888 significantly reduced soluble and insoluble forms of Aβ_{1-40,42} compared to soluble and insoluble controls (FIGS. 10 A and B, respectively).

### Example 8

### Reduction of Phosphorylated Tau Protein in Tg2576 Mice

As shown in Shytle et al., 2012, HSRx-888 (HSS-888) reduces phosphorylated tau protein in Tg2576 Mice. (FIGS. 11 A and B). Anterior quarter brain homogenates from the treated mice were analyzed by Western blot analysis.

Methodology: “Brain homogenates were obtained as previously described above. For Tau analysis, aliquots corresponding to 100 μg of total protein were separated electrophoretically using 10% Tris gels. Electrophoresed proteins were then transferred to nitrocellulose membranes (Bio-Rad, Richmond, Calif.), washed in ddH20, and blocked for 1 h at ambient temperature in Tris-buffered saline (TBS) containing 5% (w/v) non-fat dry milk. After blocking, membranes were hybridized for 1 h at ambient temperature with various primary antibodies. Membranes were then washed 3 times for 5 min each in ddH20 and incubated for 1 h at ambient temperature with the appropriate HRP-conjugated secondary antibody (1:1,000, Pierce Biotechnology, Woburn, Mass.). All antibodies were diluted in TBS containing 5% (w/v) of nonfat dry milk. Blots were developed using the Luminol reagent (Pierce Biotechnology, Woburn, Mass.). Densitometric analysis was done as previously described using a FluorS Multiimager with QUANTITY ONE™ software (BioRad, Hercules, Calif.) (Rezai-Zadeh et al., 2005).” Shytle et al., 2012

Results: Mice treated with HSRx-888 showed an 80% decrease in p-tau relative to control mice. (FIGS. 11 A and B).

### Example 9

### Reduction of Th2 Response in Cultured Microglia Cells from Tg2576 Mice

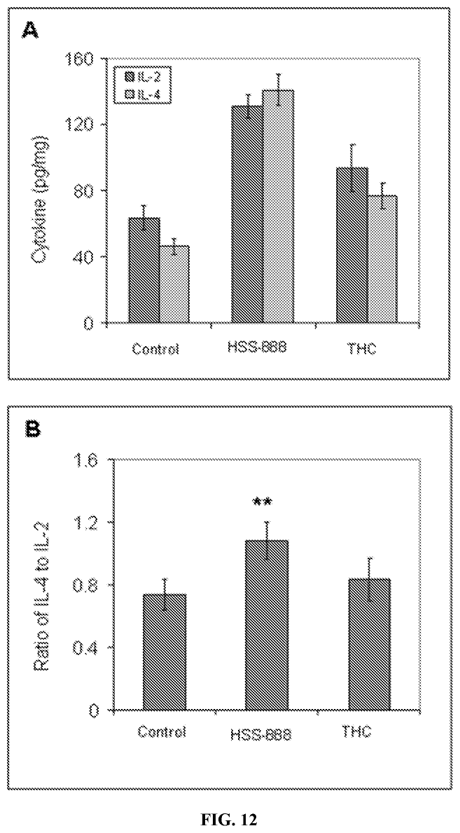

As shown in Shytle et al., 2012, HSRx-888 (HSS-888) enhances Th2 cellular immune responses, similar to what has been shown with curcumin where immune response shift from Th1 to Th2 immunity (Kang et al. 1999). (FIGS. 12 A and B). Specifically, HSRx-888 treatment increased the ratio of IL-4 to IL-2, indicating a switch from Th1 (inflammatory) to Th2 (non-inflammatory) reaction. Further, HSRx-888 treatment increased cytokines IL-2 and IL-4 indicating HSRx-888 affords microglia protection via the anti-inflammatory activity of specific cytokines. (FIGS. 12 A and B). Primary cultures of microglia were established from the mice following sacrifice and stimulated for 24 h with anti-CD 3 antibody.

Methodology: “Following sacrifice of both treatment and control groups, primary cultures of microglia were established from these mice and stimulated for 24 h with anti CD3 antibody.” Shytle et al., 2012. “As described in previous studies (Tan et al. Journal of Immunology, 1999; Tan et al. Science, 1999), cell cultured microglia were collected for measurement of cytokines by commercial cytokine ELISA kits. In parallel, cell lysates were prepared for measurement of total cellular protein. Data are represented as ng/mg total cellular protein for each cytokine produced. Cytokines were quantified using commercially available ELISAs (BioSource International, Inc., Camarillo, Calif.) that allow for detection of IL-2 and IL-4.” Shytle et al., 2012.

Results: Mice treated with HSRx-888 showed an increase in both cytokines IL-4 and IL-2 by 3 and 2 fold compared to controls, respectively (143 ng/ml and 129 ng/ml, respectively). FIG. 12 A. Further, in cells from mice treated with HSRx-888 the ratio of IL-4 to IL-2 increased from 0.73 to 1.11 in comparison to controls. FIG. 12 B. Specifically, HSRx-888 treatment increased the ratio of IL-4 to IL-2, indicating a shift from a Th1 (inflammatory) response to a Th2 (non-inflammatory) response. Increase in the ratio of Th2 response in comparison to the Th1 response is expected to decrease inflammation related to an immune response. Thus, it is expected from this data that the compositions disclosed herein possess anti-inflammatory properties.

### Example 10

### Direct Binding to Amyloid

Any proposed, non-binding mechanism of action for reduction of β-amyloid aggregation by the compositions disclosed herein does not preclude the possibility that at least one of the biomarkers disclosed herein binds amyloid and through such reduces amyloid aggregation. It was shown that biomarker 15 (BDMC) is predicted to bind βA(1-42).

Briefly, three-dimensional free-energy minimizations using Chem 3D Ultra (Cambridgesoft, Cambridge, Mass.) molecular modeling package was employed for the free-energy minimizations of biomarker 15 using the molecular mechanics two level of theory.

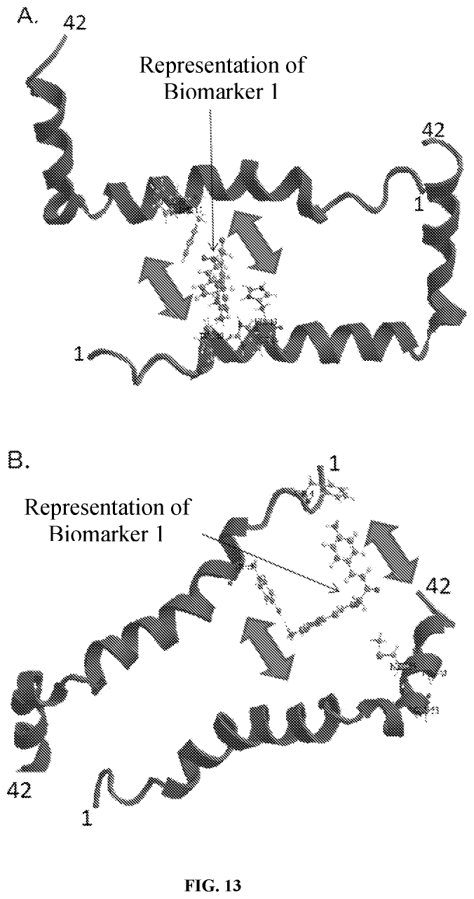

Results: Minimum free-energy modeling analysis revealed strong intermolecular interactions occur between Tyr₁₀ and biomarker 15 which allows biomarker 15 to surround His₁₃ and His₁₄ effectively preventing Phe₁₉ and Phe₂₀ from binding and forming oligomers. (FIG. 13 A). Biomarker 15 can also bind to Gly₃₃, Met₃₅, and Gly₃₇ disrupting the stabilizing intermolecular interactions of the βA(1-42) oligomers. (FIG. 13 B).

### Example 11

### Cerebrospinal Fluid Solubility

It is expected that administration of the compositions disclosed herein will provide biomarkers from such composition to the cerebrospinal fluid when administered to a subject through any means of administration. Administration may include, but is not limited to, oral, intravenous (IV), or intracoelomic (IC) administration. As support, in Example 3, biomarkers from HSRx-888 can be found in the blood serum after oral administration in humans. Further, Examples 6, 7, and 8 demonstrate that mice orally administered HSRx-888 had decreased markers for Alzheimer's disease in the brain, strongly suggesting that biomarkers from HSRx-888 made it into the cerebrospinal fluid. Also, it has been suggested that some compounds found in the serum are likely to make it into the cerebrospinal fluid (Nau et al., 2010).

Finally, to show that HSRx-888 is soluble and detectable in cerebrospinal fluid, it was shown HSRx-888 is soluble in ex vivo cerebrospinal fluid (not shown). Further, using DART-TOF, it was shown that HSRx-888 biomarkers can be detected in a mixture of HSRx-888 and ex vivo cerebrospinal fluid.

Clinical trials are currently planned to further demonstrate that oral administration of the compositions disclosed herein to a human subject will provide biomarkers from such composition to the subject's cerebrospinal fluid. See Example 12.

### Example 12

### Clinical Trials in Human Subjects

This example concerns a planned clinical trial using HSRx-888 to determine the safety and tolerability of HSRx-888 and its effects on cerebrospinal biomarkers in mild to moderate Alzheimer's disease (AD). Specifically, the study is designed to: 1) examine the safety and tolerability of two doses of the turmeric-derived nutritional supplement HSRx-888 compared to placebo in patients with mild to moderate AD; 2) determine whether curcumin is detectable in the cerebrospinal fluid of persons with AD after multiple doses of HSRX-888; and 3) examine the effects of HSRx-888 vs placebo on biomarkers of AD, including amyloid-42, tau and phospho-tau. Table 2 outlines the procedures to be followed in the study.

Methodology: 45 subjects between 50 and 90 years of age with mild to moderate AD (Mini Mental State examination (MMSE) of 14-28) receiving stable doses of an approved acetylcholinesterase inhibitor will be enrolled for the approximately 56 week study. The study will be a randomized, double-blind, placebo-controlled design.

Subjects will receive two containers of the investigational product in capsule form. Each capsule will contain 175 mg of HSRx-888 or an equivalent weight of an indistinguishable inert placebo powder. Subjects will be instructed to take two pills three times daily before meals. Missed doses should not be replaced by double doses at a later time. The Placebo arm will receive two placebo capsules three times daily. The Low DOSE HSRx-888 arm will receive one placebo capsule and one HSRx-888 capsule three times a day. The High DOSE HSRx-888 arm will receive two HSRx-888 capsules three times a day.

The total study duration will be one year and will include the following components:
1) Subjects will be randomly assigned to receive three times daily dosing of either 175 mg HSRX-888, 350 mg HSRX-888 or a matched placebo at a ratio of 1:1:1. The first 9 subjects (comprising 3 from each arm) will undergo lumbar puncture (LP) at baseline and after receiving 1 month of the study supplement. After 9 subjects have completed two LPs, an interim analysis will be carried out to determine levels of curcumin and glucuronidated curcumin in blood and cerebrospinal fluid (CSF).
2) If curcumin is confirmed to be present in the cerebrospinal fluid of the first 6 subjects who received HSRx-888, the remaining 36 subjects will be randomized to receive one of the two doses of HSRx-888 or placebo for 1 year. If no curcumin is found in CSF, or saturating amounts are found at the lower dose, or if sub-optimal amounts of curcumin are found in the CSF, then the sponsor and IRB will be informed and if deemed appropriate, a request will be made to test an additional nine subjects with adjusted doses of HSRx-888.
3) 36 additional subjects will be enrolled in the study and will undergo lumbar puncture at baseline and 6 months after randomization. Their CSF will be analyzed for free curcumin and curcumin metabolites, as well as validated Alzheimer's disease biomarkers (amyloid-42, tau and phospho-tau 181). An interim analysis of AD biomarkers will be performed when 18 subjects have completed 6 months of study product.
4) All 45 subjects will receive their randomly assigned investigational supplement or placebo for one year. Throughout that year, subjects will undergo periodic physical, neurologic and clinical assessments as well as routine laboratory tests to assess their tolerance of the investigational supplement and to obtain further information about the safety of this supplement in persons with Alzheimer's disease.

Outcomes Measured: The following outcomes will be measured in this clinical trial.

Safety: (1 year, All Subjects) Safety outcomes measured will include adverse events/serious adverse events; clinical laboratory tests (CBC, Biochemical Profile); vital signs; weight/BMI; physical and neurologic examinations; Geriatric Depression Scale (GDS); Modified Minimental State Examination (3MS); ADCS-ADL Scale; Neuropsychiatric Inventory (NPI). The inclusion of the 3MS, ADCS-ADL, GDS and NPI is to examine whether there are any adverse effects on cognition, daily function, mood or behavior associated with the study product. This component of the study may also provide data useful for powering future studies of efficacy.

6 month biomarker endpoint (6 months HSRx-888 or placebo and 2 LPs in 36 subjects): At the 6 month appointment, primary outcomes measured will include change in CSF abeta-42 after 6 months of the study supplement. Secondary outcomes measured will include change in CSF tau, phospho-tau and curcumin after 6 months administration of the study supplement.

Additional exploratory endpoints: (All subjects' CSF and blood serum) Additional endpoints that will be measured include change in level of bioactive curcumin in blood serum following the study supplement; change in level of other turmeric derived substances in blood and CSF following the study supplement; and change in levels of glucuronidated curcumin in blood and CSF following the study supplement.

First interim analysis (following 1 month of HSRx-888 or placebo and two LPs in first 9 subjects): The first interim analysis outcomes measured will include change in level of curcumin in cerebrospinal fluid after one month and change in level of curcumin in blood after one month.

Second Interim Analysis (following 6 months of HSRx-888 or placebo and two LPs in 18 subjects): The second interim analysis outcomes measured will include change from baseline in level of bioactive curcumin in cerebrospinal fluid after six months of three times daily dosing; change from baseline in curcumin concentration in blood after six months of three times daily dosing; and change from baseline in CSF abeta-42/tau and phospho-tau.

Statistical Analysis: Descriptive statistics will be used to characterize the study population as a whole and to check for imbalances across study arms. The Kruskal-Wallis Test or a similar non-parametric test will be used to assess significance of differences from baseline to three months in cerebrospinal curcumin levels for the interim analysis of first 9 subjects. For the biomarker analysis after 6 months of HSRx-888 or placebo, an ANOVA will be performed using Last Observation Carried Forward methodology for imputation of missing data. In the event that there are significant differences in age, baseline MMSE or other demographic measures across study arms, an ANCOVA will be employed to adjust for the imbalance. Safety outcomes will be tabulated for the study as a whole and broken down by study arm. Comparisons will be made between subjects receiving HSRx-888 vs placebo in term of frequency and severity of AEs.

TABLE 2
Table of Procedures

| | Description: | | | | |
|---|---|---|---|---|---|
| | Screening visit | Baseline | 1st Follow-up | 2nd Follow-up | Final |
| | (0) | Visit | Visit | Visit | Visit |
| | Week: | | | | |
| | −4 | 0 | 4 | 24 | 52 |
| | Visit # | | | | |
| Procedure | 1 | 2 | 3 | 4 | 5 |
| Informed consent | X | | | | |
| Medical History/AD Dx | X | | | | |
| Confirmation | | | | | |
| Minimental State Exam | X | | | | |
| (MMSE) | | | | | |
| Geriatric Depression Scale | X | | X | X | X |
| (GDS) | | | | | |
| Modified Hachinski Score | X | | X | X | X |
| Vital Signs | X | | X | X | X |
| Physical and Neurologic Exam | X | | X | X | X |
| Safety Laboratory tests * | X | | X | X | X |
| Coagulation profile ** | X | | X | | |
| EKG | X | | | | |
| MRI or CT scan ** | X | | | | |
| Medication review | X | X | X | X | X |
| Fluoro-guided lumbar | | All | First 9 subjects | Next 36 subjects | |
| puncture | | Subjects | only | only | |
| Blood for curcumin level | | X | X | X | X |
| APOE Genotype | | X | | | |
| Modified Minimental exam | | X | X | X | X |
| (3MS) | | | | | |
| ADCS-Activities of Daily Living | | X | X | X | X |
| (ADL) | | | | | |
| Neuropsychiatric Inventory | | X | X | X | X |
| (NPI) | | | | | |
| Adverse event review | | X | X | X | X |
| Supplement compliance | | | X | X | X |
| (missed doses) | | | | | |

* Safety Labs: Complete blood count with platelet count, Complete metabolic profile, TSH, B12, Urinalysis, HgA1C
** Coagulation profile: Protime (PT), Prothrombin Time (PTT) and INR
*** MRI or CT scan within past 6 months c/w AD and no contraindications to LP. Otherwise a new scan required.

### Example 13

### Anti-Oxidant Capacity

This example concerns data obtained regarding the anti-oxidant capacity of HSRx-888 using a 2,2-diphenyl-1-picrylhydrazyl (DPPH) methodology.

Methodology: Briefly, stock solutions of HSRx-888 were dissolved in neat ethanol (USP) and/or Tris-HCL buffer (pH 7.4). Serial dilutions were prepared and 100 μL of each dilution were added to duplicate wells in a 96-well plate. Positive control wells and appropriate sample wells contained 100 μL Tris-HCl buffer and 100 μL of 500 μM DPPH in neat ethanol. Tris-HCl buffer was added to the blank sample wells yielding 200 μL total volumes in each blank well. The plate was shaken for 20 min in the dark at room temperature and the absorbance was measured at 517 nm using a BioTek Synergy microplate reader (Biotek, Winooski, Vt.). The DPPH radical-scavenging activity was defined as the difference in absorbance between blank and DPPH containing sample wells relative to DPPH positive controls.

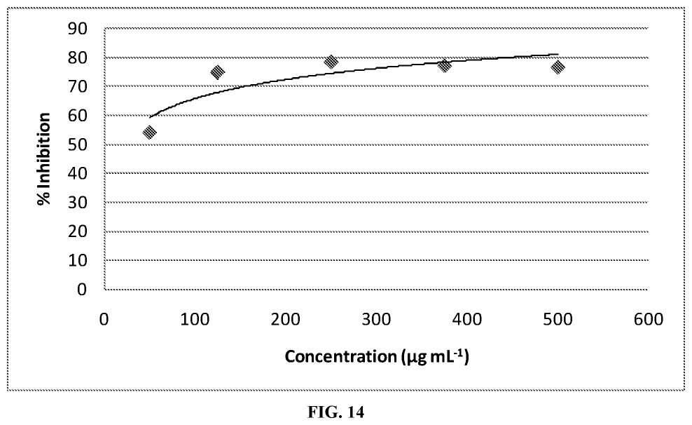

Results: It was determined that HSRx-888 inhibited 2-diphenyl-1-picrylhydrazyl radical (DPPH) in a dose dependent manner. The HSRx-888 IC₅₀ value is 19.2 μg mU⁻¹ (R²=0.731, N=10). (FIG. 14)

### Example 14

### Anti-Inflammatory Properties

This example concerns data obtained regarding the inhibition of COX1, COX2, and 5LOX. This data shows that HSRx-888 in an anti-inflammatory.

### COX1 and COX2 Assays

Methodology: Briefly, all reagents and solutions were prepared according to the protocols established by Cayman Chemicals (Ann Arbor, Mich.) for the COX-1 and COX-2 inhibition assays. Two procedures were utilized to assess the COX1/2-specific and non-specific activities.

Prostaglandin Production Inhibition: Turmeric extracts were dissolved in neat dimethylsulfoxide (DMSO), and then diluted in reaction buffer to a final DMSO concentration of 1% (v/v). Reactions were run with COX-1 (ovine) or COX-2 (human recombinant) enzymes in the presence of Heme. Wells containing turmeric extracts, 100% enzyme activity, background wells (heat inactivated enzymes), and the appropriate blanks were prepared. Solutions were placed in a 37° C. incubator for 15 min. prior to running the reaction. Arachidonic acid was added and the reaction proceeded for 2 min. The reaction was stopped by addition of 1 M HCl. The Prostaglandin F₂ product was quantified using EIA.

Quantification of Prostaglandin with EM: The assay plate (EIA) was provided in the Cayman Chemicals screening kit. Aliquots (50 μL) of the reaction products (PGF₂) from prostaglandin production were added to their respective wells. Total activity and blank wells received 150 μL of EIA buffer, non-specific binding wells received 100 μL of EIA buffer, and maximum binding wells received 50 μL of EIA buffer. COX 100% activity wells, non-specific binding, background, maximum binding, standards, and turmeric extract wells received 50 μL of tracer. COX 100% activity, background, maximum binding, standards, and turmeric extract wells also received 50 μL of antiserum. The EIA plate reactions were run for 18 h at room temperature. Plates were washed with wash buffer and 200 μL Ellman's Reagent was added to all wells, followed by 5 μL of tracer to the total activity well. The color development was quantified by absorbance at 409 nm using a BioTek Synergy microplate reader.

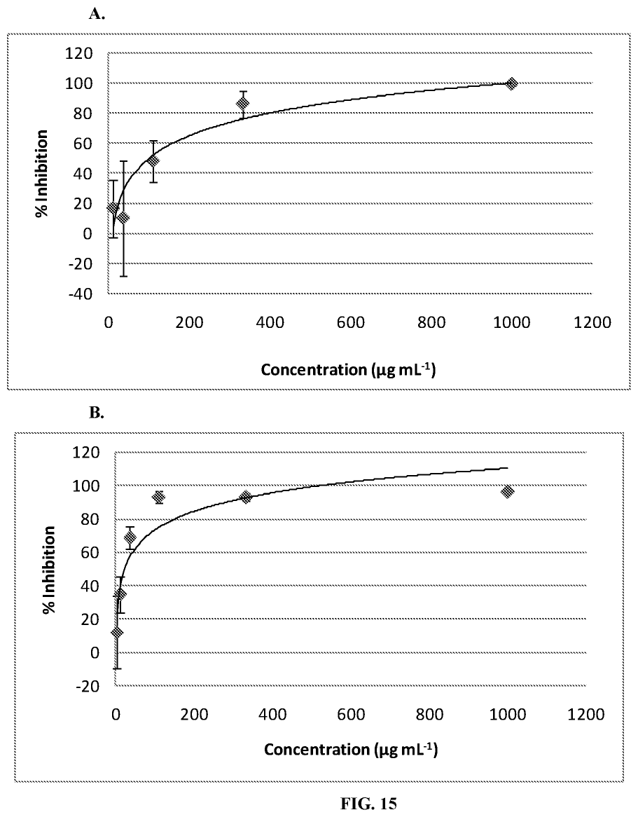

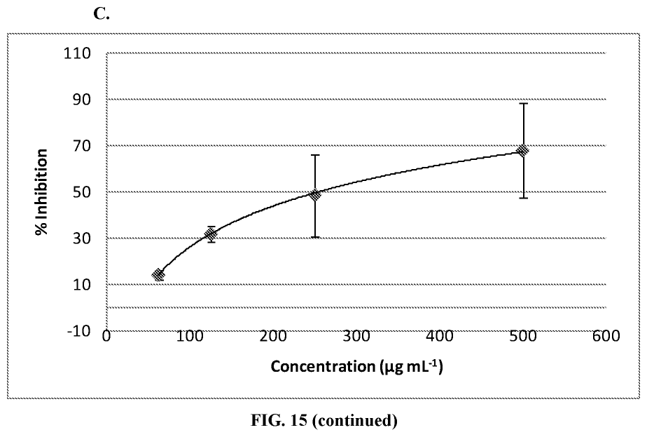

Results: It was determined that HSRx-888 inhibited COX1 and COX2 in a dose dependent manner. The HSRx-888 IC₅₀ values are 100.6 μs mL⁻¹ (R²=0.907, N=36) for COX1 (FIG. 15 A) and 23.0 μg mL⁻¹ (R²=0.874, N=24) for COX2 (FIG. 15 B).

### 5-LOX Assay

Methodology: Briefly, 5-Lipoxygenase (5-LOX) activity was determined by monitoring purified potato 5-LOX according to the manufacturer's protocol for the Lipoxygenase Inhibitor Screening Assay Kit (Cayman Chemical, Ann Arbor, Mich.). Turmeric extracts were dissolved in neat DMSO, and serially diluted in reaction buffer to a final DMSO concentration of 1% (v/v) in all wells. Reactions were run according to the manufacturer's specifications and controls were run to establish that the 1% (v/v) DMSO did not interfere with the reactions. The 5-Lipoxygenase activity inhibition was quantified by measuring the absorbance at 495 nm using a Biotek Synergy plate reader (Winooski, Vt.) after addition of chromagen imaging reagent.

Results: It was determined that HSRx-888 inhibited 5LOX in a dose dependent manner. The HSRx-888 IC₅₀ value is 256.3 μs mL⁻¹ (R²=0.999, N=8) for 5LOX (FIG. 15 C).

### Example 15

### Synergy

As previously noted, experimental results herein suggest synergism between the biomarkers disclosed herein. Further, because of the predicted method of action of the biomarkers disclosed herein, it is believed that the biomarkers will act synergistically with other compounds that act through a separate mechanism to treat or prevent Alzheimer's disease, protein misfolding/aggregation disease and conditions, and/or inflammation. To further confirm such synergism and determine synergism with other compounds/compositions, one or more of the biomarkers disclosed herein can be tested in combination with one or more of the other biomarkers disclosed herein, and/or one or more drugs and/treatments. Combination studies can show competitive, additive, or synergistic interactions for treatment and/or prevention of disease and/or conditions and/or the symptoms thereof in cell culture, animal studies, human studies, etc. Non-limiting examples of studies can include those described above and herein as well as those known to one of skill in the art. As a non-limiting example, the combination of HSRx-888 and NSAIDs, NMDA receptor antagonist, and/or acetylcholinesterase inhibitors may be tested.

A non-limiting example of a combination assay that can be performed to determine the competitive, additive, or synergistic interactions of a combination can utilize an interaction matrix commonly used to look at drug interactions and synergy. In one instance, the interaction matrix is used in a prevention or treatment study of Alzheimer's disease, protein misfolding, protein aggregation, or inflammation in cell culture. Briefly, the experiment can have 25 samples: 4 with a first test compound/composition (such as HSRx-888) alone, 4 with a second test compound/composition alone, 1 with no chemistries, and the remaining 16 can be combinations of the first and second test compounds/compositions. 1:4 dilutions of the first test compound/composition from a starting concentration (such as 1 mg/nil for HSRx-888) and 1:4 dilutions of the second test compound/composition from a starting concentration can be tested. The ability to decrease inflammation markers, decrease amyloid secretion, decrease amyloid aggregation, decrease phosphorylation of tau, etc. can occur in the constant presence of the inhibitory compounds. In this way, the experiment simulates a patient while on prophylactic treatment and tests prevention of disease onset by the first test compound/composition alone, the second test compound/composition alone, and the combination of the two at a range of concentrations. The data can be analyzed with the methodology of Berenbaum to determine competitive, additive, or synergistic interactions. (Berenbaum 1977).

### Example 15

### Expected Treatment and/or Prevention of Multiple Neurological Disorders

The combinations disclosed herein provide benefits in treatment and/or prevention of multiple diseases, disorders, and conditions based on the benefits disclosed herein and the benefits of treatments with curcumin, including those demonstrated in non-human models and in vitro. As demonstrated herein, combinations of biomarkers disclosed herein can increase the uptake of curcumin in human subjects, are soluble in cerebrospinal fluid, possess anti-inflammatory properties, possess anti-oxidant capacity, and possess an ability to decrease protein degeneration and/or misfolding. Based on these properties, among others, it expected that the combinations disclosed herein can provide increased amounts of bioavailable curcumin, increased anti-inflammatory benefits, increased anti-oxidant benefits, decreased protein degeneration benefits, and decreased protein misfolding benefits to human subjects.

For at least those reasons described and demonstrated herein, the combinations disclosed herein provide benefits in treatment and/or prevention for neurological disorders, diseases, and conditions that include, but are not limited to, degenerative/protein misfolding disorders, cerebrovascular diseases, inflammatory diseases, trauma/closed head injuries, epilepsies, and/or neoplasms. Non-limiting examples of degenerative/protein misfolding disorders include Alzheimer's, Parkinson's, Lewy body, frontotemporal degeneration, progressive supranuclear palsy, amyotrophic lateral sclerosis, multisystem atrophy, cerebral amyloidosis, spinocerebellar atrophy. Non-limiting examples of cerebrovascular diseases include ischemic stroke, reperfusion injury, and cerebral vasospasm. Non-limiting examples of inflammatory diseases include multiple sclerosis and CNS lupus. Non-limiting examples of trauma/closed head injuries include concussions, contusions, and chronic traumatic encephalopathy. Non-limiting examples of epilepsies include generalized seizure disorders and partial seizure disorders. Non-limiting examples of neoplasms include metastatic and primary CNS tumors.

Further, as disclosed and demonstrated herein, combinations of biomarkers disclosed herein can increase the uptake of curcumin in human subjects. For at least this reason, and those described above, the combinations of biomarkers and curcumin disclosed herein will provide to human subjects the benefits associated with curcumin demonstrated in in vitro, in vivo, and/or clinical trials.

All of the compositions and/or methods disclosed and claimed herein can be made and executed without undue experimentation in light of the present disclosure. While the compositions and methods of this invention have been described in terms of particular embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

Diseases and Conditions, Alzheimer's disease: Mayo Clinic. Jun. 17, 2014. [cited 2015 October 16]. http://www.mayoclinic.org/diseases-conditions/alzheimers-diseasefbasics/definition/con-20023871
Beevers C S, et al. (2011), Pharmacological and clinical properties of curcumin. Botanics: Targets and Therapy 1: 5-18.
Berenbaum M C. (1977) Synergy, additivism and antagonism in immunosuppression. A critical review. Clin Exp. Immunol. 28(1): 1-18.
Bishnoi M., et al. (2005) Protective effects of nimesulide (COX Inhibitor), AKBA (5-LOX Inhibitor), and their combination in aging-associated abnormalities in mice. Methods and Findings in Experimental and Clinical Pharmacology 27(7):465-470.
Cody R B, Laramee J A and Durst H D. (2005) Versatile new ion source for the analysis of materials in open air under ambient conditions. Analytical Chemistry 77:2297-2302.
Ellisdon A M, Bottomley S P. (2004) The role of protein misfolding in the pathogenesis of human diseases. IUBMB Life 56(3):119-123.
Garcia-Alloza M, Robbins E M, Zhang-Nunes S X, Purcell S M, Betensky R A, Raju S, et al. (2006) Characterization of amyloid deposition in the APPswe/PS1 dE9 mouse model of Alzheimer disease. Neurobiol Dis 24(3):516-524.
Hoozemans J J, et al. (2008) Cyclooxygenase-1 and -2 in the different stages of Alzheimer's disease pathology. Current Pharmaceutical Design 14(14):1419-1427.
Johnson-Wood K, et al. (1997) Amyloid precursor protein processing and A β42 deposition in a transgenic mouse model of Alzheimer's disease. Proceedings of the National Academy of Sciences USA 94(4):1550-1555.
Kang B Y, et al. (1999) Curcumin inhibits Th1 cytokine profile in CD4+ T cells by suppressing interleukin-12 production in macrophages. British Journal of Pharmacology 128(2):380-384.
LeVine H. (1993) Thioflavine T interaction with synthetic Alzheimer's disease beta-amyloid peptides: detection of amyloid aggregation in solution. Protein Sc. 2:404-410.
Lim G P, Chu T, Yang F, et al. (2001) The curry spice curcumin reduces oxidative damage and amyloid pathology in an Alzheimer transgenic mouse. J Neurosci. 21(21):8370-7.
Ma Q L, Zuo X, Yang F, et al. (2013) Curcumin suppresses soluble tau dimers and corrects molecular chaperone, synaptic, and behavioral deficits in aged human tau transgenic mice. J Biol Chem. 288(6):4056-65.
Marambaud P, Zhao H, Davies P. (2005) Resveratrol promotes clearance of Alzheimer's disease amyloid-beta peptides. J Biol Chem 280:37377-37382.
Moore S A, Huckerby T N, Gibson G L, Fullwood N J, Turnbull S, Tabner B J, et al. (2004) Both the D-(+) and L-(−) enantiomers of nicotine inhibit Abeta aggregation and cytotoxicity. Biochemistry 43: 819-826.
Nau R, Sörgel F, Eiffert H. (2010) Penetration of Drugs through the Blood-Cerebrospinal Fluid/Blood-Brain Barrier for Treatment of Central Nervous System Infections. Clinical Microbiology Reviews. 23:858-883.
Ng T P, Chiam P C, Lee T, Chua H C, Lim L, Kua E H. (2006) Curry consumption and cognitive function in the elderly. Am J Epidemiol. 164:898-906.
Obregon D F, Rezai-Zadeh K, Bai Y, Sun N, Hou H, Ehrhart J, et al. (2006) ADAM10 activation in required for green tea (−)-epigallocatechin-3-gallate-induced alpha-secretase cleavage of amyloid precursor protein. J Biol Chem 281: 16419-16427.
Rezai-Zadeh K, et al. (2005) Green tea epigallocatechin-3-gallate (EGCG) modulates amyloid precursor protein cleavage and reduces cerebral amyloidosis in Alzheimer transgenic mice. J Neurosci 25(38):8807-8814.
Rezai-Zadeh K, et al. (2008) Green tea epigallocatechin-3-gallate (EGCG) reduces b-amyloid mediated cognitive impairment and modulates tau pathology in Alzheimer transgenic mice. Brain Research 1214:177-187.
Ringman J M, Frautschy S A, Cole G M, Masterman D L, Cummings J L. (2005) A potential role of the curry spice curcumin in Alzheimer's disease. Curr Alzheimer Res. 2(2):131-136.
Ringman J M, Frautschy S A, Teng E, et al. (2012) Oral curcumin for Alzheimer's disease: tolerability and efficacy in a 24-week randomized, double blind, placebo-controlled study. Alzheimers Res Ther. 4(5):43.
Shytle R D, Bickford P C, Rezai-zadeh K, Hou L, Zeng J, Tan J, Sanberg P R, Sanberg C D, Roschek B Jr., Fink R C, Alberte R S. (2009) Optimized turmeric extracts have potent anti-amyloidogenic effects. Curr Alzheimer Res. 6(6):564-571.
Shytle R D, Tan J, Bickford P C, Rezai-Zadeh K, Hou L, Zeng J, Sanberg P R, Sanberg C D, Alberte R S, Fink R C, Roschek B Jr. (2012) Optimized turmeric extract reduces 13-Amyloid and phosphorylated Tau protein burden in Alzheimer's transgenic mice. Curr Alzheimer Res. 9(4):500-6.
Silverman E S, Drazen J M. (1999) The biology of 5-lipoxygenase: function, structure, and regulatory mechanisms. Proceedings of the Association of American Physicians 111(6):525-536.
Steinhilber D., Hofmann B. (2013) Recent Advances in the Search for Novel 5-Lipoxygenase Inhibitors. Basic Clin Pharmacol Toxicol. 114(1):70-77
Tan J, et al. (1999) Ligation of microglial CD40 results in p44/42 mitogen-activated protein kinase-dependent TNF-alpha production that is opposed by TGF-beta 1 and IL-10. Journal of Immunology 163(12):6614-6621.
Tan J, et al., (1999) Microglial activation resulting from CD40-CD40L interaction after beta-amyloid stimulation. Science 286(5448):2352-2355.
Tan J, Town T, Crawford F, Mori T, DelleDonne A, Crescentini R, Obregon D, Flavell R A, Mullan M J. (2002) Role of CD40 ligand in amyloidosis in transgenic Alzheimer's mice. Nat Neurosci 5:1288-1293.
Yang F, Lim G P, Begum A N, et al. (2005) Curcumin inhibits formation of amyloid beta oligomers and fibrils, binds plaques, and reduces amyloid in vivo. J Biol Chem. 280(7):5892-5901.

1.-20. (canceled)

21. A composition comprising a combination of:
biomarker 1 having an accurate mass of 120.094 amu and having a relative abundance of at least 2.17%;
biomarker 2 having an accurate mass of 134.110 amu and having a relative abundance of at least 0.31%;
biomarker 6 having an accurate mass of 200.157 amu and having a relative abundance of at least 0.47%;
biomarker 12 having an accurate mass of 232.146 amu and having a relative abundance of at least 2.38%;
biomarker 3 having an accurate mass of 150.104 amu and having a concentration of at least 0.04% by weight;
biomarker 4 having an accurate mass of 176.120 amu and having a relative abundance of at least 0.96%;
biomarker 5 having an accurate mass of 192.091 amu and having a relative abundance of at least 1.74%;
biomarker 7 having an accurate mass of 202.172 amu and having a relative abundance of at least 0.87%;
biomarker 8 having an accurate mass of 204.188 amu and having a relative abundance of at least 0.30%;
biomarker 9 having an accurate mass of 216.151 amu and having a relative abundance of at least 10.75%;
biomarker 10 having an accurate mass of 218.203 amu and having a relative abundance of at least 4.00%;
biomarker 11 having an accurate mass of 220.183 amu and having a relative abundance of at least 0.72%;
biomarker 13 having an accurate mass of 234.162 amu and having a relative abundance of at least 3.52%;
biomarker 14 having an accurate mass of 256.240 amu and having a relative abundance of at least 0.25%;
biomarker 15 having an accurate mass of 308.105 amu and having a concentration of at least 1.50% by weight;
biomarker 16 having an accurate mass of 338.115 amu and having a concentration of at least 1.67% by weight;
biomarker 18 having an accurate mass of 372.157 amu and having a concentration of at least 0.88% by weight;
biomarker 19 having an accurate mass of 450.261 amu and having a relative abundance of at least 0.61%; and
curcumin and/or a functional derivative of curcumin,
wherein each biomarker is found in Curcuma longa, and
wherein the relative abundance is relative to 25 mg/ml salicylic acid spiked in 0.5 mg/ml of the composition dissolved in ethanol.

22. The composition of claim 21, wherein the composition further comprises at least one acetylcholinesterase inhibitor, at least one N-methyl-D-aspartate (NMDA) receptor antagonist, and/or at least one anti-inflammatory drug.

23. The composition of claim 22, wherein the at least one acetylcholinesterase inhibitor is donepezil, tacrine, galantamine, rivastigmine, salts thereof, or any combination thereof, the at least one N-methyl-D-aspartate (NMDA) receptor antagonist is memantine, and/or the at least one anti-inflammatory drug is a nonsteroidal anti-inflammatory drug.

24. The composition of claim 21, wherein the composition is formulated for intranasal administration, topical application, administration through injection, and/or oral administration.

25. The composition of claim 21, wherein the composition further comprises at least one turmerone and has a weight ratio of curcumin and/or an analog thereof to turmerones of between 0.5 to 0.9.

26. The composition of claim 21, wherein the composition is formulated to provide at least 10 mg of curcumin and/or functional derivative thereof into the serum of a human administered the composition and/or to provide at least 1 mg of curcumin and/or functional derivative thereof into the cerebrospinal fluid of a human administered the composition.

27. The composition of claim 21, further comprising an imaging agent in the composition and/or covalently bound to at least one of the biomarker(s) 1 through 16, 18, or 19.

28. A composition comprising curcumin and/or a functional derivative of curcumin and one or more of:
biomarker 1 having an accurate mass of 120.094 amu and having a relative abundance of at least 2.17%;
biomarker 2 having an accurate mass of 134.110 amu and having a relative abundance of at least 0.31%;
biomarker 6 having an accurate mass of 200.157 amu and having a relative abundance of at least 0.47%;
biomarker 12 having an accurate mass of 232.146 amu and having a relative abundance of at least 2.38%;
biomarker 3 having an accurate mass of 150.104 amu and having a concentration of at least 0.04% by weight;
biomarker 4 having an accurate mass of 176.120 amu and having a relative abundance of at least 0.96%;
biomarker 5 having an accurate mass of 192.091 amu and having a relative abundance of at least 1.74%;
biomarker 7 having an accurate mass of 202.172 amu and having a relative abundance of at least 0.87%;
biomarker 8 having an accurate mass of 204.188 amu and having a relative abundance of at least 0.30%;
biomarker 9 having an accurate mass of 216.151 amu and having a relative abundance of at least 10.75%;
biomarker 10 having an accurate mass of 218.203 amu and having a relative abundance of at least 4.00%;
biomarker 11 having an accurate mass of 220.183 amu and having a relative abundance of at least 0.72%;
biomarker 13 having an accurate mass of 234.162 amu and having a relative abundance of at least 3.52%;
biomarker 14 having an accurate mass of 256.240 amu and having a relative abundance of at least 0.25%;
biomarker 15 having an accurate mass of 308.105 amu and having a concentration of at least 1.50% by weight;
biomarker 16 having an accurate mass of 338.115 amu and having a concentration of at least 1.67% by weight;
biomarker 18 having an accurate mass of 372.157 amu and having a concentration of at least 0.88% by weight; and
biomarker 19 having an accurate mass of 450.261 amu and having a relative abundance of at least 0.61%,
wherein each biomarker is found in Curcuma longa, and
wherein the relative abundance is relative to 25 mg/ml salicylic acid spiked in 0.5 mg/ml of the composition dissolved in ethanol.

29. The composition of claim 28, wherein the composition further comprises at least one acetylcholinesterase inhibitor, at least one N-methyl-D-aspartate (NMDA) receptor antagonist, and/or at least one anti-inflammatory drug.

30. The composition of claim 29, wherein the at least one acetylcholinesterase inhibitor is donepezil, tacrine, galantamine, rivastigmine, salts thereof, or any combination thereof, the at least one N-methyl-D-aspartate (NMDA) receptor antagonist is memantine, and/or the at least one anti-inflammatory drug is a nonsteroidal anti-inflammatory drug.

31. The composition of claim 28, wherein the composition is formulated for intranasal administration, topical application, administration through injection, and/or oral administration.

32. The composition of claim 28, wherein the composition further comprises at least one turmerone and has a weight ratio of curcumin and/or an analog thereof to turmerones of between 0.5 to 0.9.

33. The composition of claim 28, wherein the composition is formulated to provide at least 10 mg of curcumin and/or functional derivative thereof into the serum of a human administered the composition and/or to provide at least 1 mg of curcumin and/or functional derivative thereof into the cerebrospinal fluid of a human administered the composition.

34. The composition of claim 28, further comprising an imaging agent in the composition and/or covalently bound to at least one of the biomarker(s) 1 through 16, 18, or 19.

35. A method of treating a subject at risk for and/or having a neurological disease, disorder, and/or condition, the method comprising administering the composition of claim 21 to the subject, wherein the neurological disease, disorder, and/or condition is ameliorated in the subject and/or the onset is delayed in comparison to the expected onset of the neurological disease, disorder, and/or condition if the patient had not been treated.

36. The method of claim 35, wherein the neurological disease, disorder, and/or condition is: a degenerative/protein misfolding disease, disorder, and/or condition; a cerebrovascular disease, disorder, and/or condition; an inflammatory disease, disorder, and/or condition; a trauma/closed head injury; an epilepsy; and/or a neoplasm.

37. The method of claim 35, wherein the neurological disease, disorder, and/or condition is Alzheimer's disease, Parkinson's disease, a Lewy body disease, frontotemporal degeneration, progressive supranuclear palsy, amyotrophic lateral sclerosis, multisystem atrophy, cerebral amyloidosis, spinocerebellar atrophy, ischemic stroke, reperfusion injury, cerebral vasospasm, multiple sclerosis, CNS lupus, a concussion, a contusion, chronic traumatic encephalopathy, a generalized seizure disorder, a partial seizure disorder, a metastatic tumor, and/or a primary CNS tumor.

38. The method of claim 35, wherein the neurological disease, disorder, and/or condition is Alzheimer's disease.

39. The method of claim 35, wherein amyloid aggregation is decreased, amyloid secretion is decreased, tau level is decreased, phosphorylated tau level is decreased, phosphorylation of tau is decreased, protein misfolding is decreased, protein aggregation is decreased, reactive oxygen species levels are decreased, free radical levels are decreased, neuro-inflammation is decreased, IL-4 to IL-2 ratio is increased, cognition is increased, and/or uptake of curcumin and/or a functional derivative thereof into a subject is increased when compared to the uptake of curcumin and/or a functional derivative thereof without any of biomarkers 1 through 16, 18, and/or 19.

40. A method of increasing curcumin and/or functional derivative thereof uptake into the serum and/or cerebrospinal fluid of a subject, the method comprising administering the composition of claim 21 to the subject, wherein curcumin and/or functional derivative thereof uptake is increased in comparison to administration of curcumin and/or functional derivative thereof without any of biomarkers 1 through 16, 18, or 19.
